(19) United States
(12) Patent Application Publication
(10) Pub. No.: US 2022/0168221 A1
(43) Pub. Date: Jun. 2, 2022

(54) Methods and Compositions for Microdosing, Simulating Extended Release Formulations

(71) Applicant: David J. Larwood, Woodside, CA (US)

(72) Inventor: David J. Larwood, Woodside, CA (US)

(21) Appl. No.: 17/395,254

(22) Filed: Aug. 5, 2021

Related U.S. Application Data
(60) Provisional application No. 63/061,789, filed on Aug. 5, 2020.

(51) Int. Cl. A61K 9/08; A61K 31/506

(52) U.S. Cl. CPC A61K 9/08; A61K 31/506

(57) ABSTRACT

This application is directed to a method of microdosing by dissolving drug in water and permitting a human or animal to consume the drug during waking hours. Metering for an animal is the amount of water they drink, in the pattern as consumed. By setting drug concentration so that a target dose will be consumed in a typical amount of daily drinking water, the dose is spread out into a series of smaller doses over time. For a human, this can be metered more specifically and thoughtfully. A mechanism to make this convenient for humans is provided. A model of blood levels is described. The clinical benefits are greater than predicted from extrapolating from a daily dose to dosing twice a day to a projected 6-12 times a day with the microdosing method.

### FIELD OF THE INVENTION

The present invention relates generally to a method of utilizing the pharmacokinetic properties of compounds and selective formulation to simulate modified pharmacokinetic properties of the compound, particularly for selectively and slowly administering a drug with generally rapid clearance, thereby simulating how a sustained release formulation of the compound could act in an animal model. This invention refers in particular to a finely divided dosing schedule and mechanism to provide more consistent blood drug levels and more effective utilization of a provided drug. This invention enables both a more consistent level of drug in the blood and a convenient method to ascertain a just-sufficient effective level of drug for a therapeutic regime.

The present invention relates generally to a method of using compounds capable of inhibiting chitin synthase to treat fungal infections in mammals. In one preferred embodiment, the present invention is directed to the use of a class of compounds known as nikkomycins to treat infections of Coccidioides spp. in mammals.

This application claims priority from a provisional application No. 63/061,789 filed Aug. 5, 2020, of the same title with the same inventor. This application claims priority as well from a provisional application 63/174,537 filed April 13 entitled “METHODS AND COMPOSITIONS FOR TREATING DISSEMINATED COCCIDIOIDOMYCOSIS WITH NIKKOMYCIN Z MICRO DOSING,” and provisional application 63/175,594, filed Apr. 16, 2021, entitled Methods and Compositions for Treating Central Nervous System Coccidioidomycosis with Nikkomycin Z Micro Dosing.” All of these are incorporated here by reference.

### BACKGROUND OF THE INVENTION

Many drugs with potential clinical benefit are a challenge to formulate because they clear the body rapidly, requiring frequent or high dosing to maintain a threshold drug level. Patients generally do not like dosing frequency greater than twice a day. Many will put up with brief durations of dosing nominally every six hours (“QID”, four times a day). Patient enthusiasm for such frequent dosing fades as the duration of therapy gets longer, and compliance becomes more challenging. Ten days is common enough in antibiotic therapy, and tolerated by many patients, but months of QID dosing quickly becomes tedious and even highly motivated compliance suffers from increasing missed or delayed doses. Even with great care, a strict q. 6-hour schedule interferes with a normal sleep cycle of 8 hours. (q. for “Qua” which means “every” such as every NN hours, q. 6 hours is 4 times a day, or QID). People will follow this if the therapy is important enough, even though the schedule is disruptive.

The therapeutic index (TI) is an important dosing parameter. The “TI” is the level that is safe to take (safety level), divided by the level that is required for a desired effect (effect level). For drugs that have significant toxicity, a common attribute of traditional anti-cancer drugs, it is not uncommon for the safety level to be only slightly above the minimum effect level, by as little as 10-25% (110-125% of minimum effect level). For such drugs, it is important to manage attentively dosing levels and frequency to stay below the safety limit. Many drugs, such as many anti-cancer drugs, have such a small difference between a required minimum and a dangerous level that the drugs are best and sometimes only administered by IV infusion under attentive control. For other drugs, the TI may be a factor of 2, permitting more latitude in dosage regiments. For some drugs the TI may be significantly higher.

This is further complicated by individual patient sensitivity, other drugs they may be taking, and comorbidities. These may lower the safety level, or may change the effective level up or down, and perhaps for more than one drug in these combinations. This is still further complicated by drug-drug interactions (DDI), such as taking a drug 1 that is metabolized by the liver and a second drug 2 that modifies liver function, thereby changing the liver metabolism of drug 1 and changing the blood drug levels of drug 1.

Reviewing basic pharmacokinetics (PK) briefly, in general, the momentary blood drug level of a drug reflects a balance of drug input and drug clearance at that moment, a balance that is typically not in stasis with periodic oral dosing. In a typical example of IV infusion, all drug reaches the blood stream, at the rate of infusion, so the input rate can be fixed and constant. Drug clearance reduces blood drug level at a half-life pattern, offset against the infusion. Clearance can be complex, with multiple or compound half lives. Nikkomycin Z, as for many drugs, as at least mostly a simple, single half-life, clearing half the drug from the blood in that time period.

Typically, when infusing at a constant rate, after one clearance half-life the blood drug level will reach 50% of what will be the steady state level. For each successive such time period, the blood drug level will rise to 50% of the delta from steady state, so in successive time periods the blood drug level is 50%, 75%, then 87.5% by the third time period, then 94%, 97%, and then 98% of the steady state blood drug level by the 6th time period, effectively at steady state. When the infusion is stopped, the blood drug level drops in the same pattern of 50% in each half-life, to 50%, 25%, then 12.5% of the steady state level by the third time period.

For a drug with a clearance half-life of 2.5 hours, such as for nikZ, at 7.5 hours (3 half-lives), plus 30 minutes the drug level has dropped to about 10% of the maximum. Reinforcing that with a new dose every 8 hours gives a pattern of blood drug level swings of about 90% of peak (approximately, there is some uptake time after an oral dose, and for the moment neglecting cumulative effects). If the therapeutic effect level is just sufficient in the blood at 8 hours after dosing, this means that for most of 8 hours the blood drug levels were higher than the therapeutic effect level, peaking about 10 times higher than therapeutic levels, and considerably more drug was ingested than needed if compared to an idealized “just enough” dosing regimen such as an IV infusion. For a shorter half-life drug taken at 8 hour intervals, the 8 hour levels will be still lower and the peak is a still larger multiple of the minimum blood drug level. For a drug with a half-life of about 1.4 hours, peaking at 1 hour after dosing, 8 hours covers 5 half-lives and the level will have dropped to about 2% of the peak, close to negligible.

The model gets a bit more complicated in that taking a second dose when there is some first residual drug level will drive blood drug levels to a second peak, higher than the first, additive by roughly the amount of the first residual. When waiting six half-lives before giving a new dose, the residual will be negligible. Giving a new dose after two half-lives, the first residual level will be about 25% of the first peak, so the second peak will be roughly that amount higher, and the second residual level will be higher than the first residual, but not simply double. As a crude approximation, the serial additive effect will be less by the third dose and approaching consistent repeatability by the 6th dose, much as in the continuous infusion model. Note that dosing in this pattern will also somewhat raise the minimum blood drug level (Cₘᵢₙᵢₘᵤₘ or Cₘᵢₙ). For drugs with this pattern, it is common to begin therapy with a loading dose (larger than typical) to raise blood drug levels overall, then settle down into the pattern discussed, rather than rising to it over several repeated dosings.

If a drug clears rapidly, taking a drug every 12 hours may easily lead to a range of blood drug levels that vary by a factor of 3 or 10 or approaching infinity (full peak to almost zero).

Further, if a drug acts well at the effective level, but no additional benefit is conferred by higher levels, then giving high doses to assure surpassing the effective level means that some and perhaps considerable drug only floods the system, conferring no additional therapeutic benefit. Keeping the blood drug level minimally above the effective level will improve utilization of the drug. This is particularly important for a drug that is hard to get, due to cost or limited supply. Yet another problem with high drug levels is higher patient exposure, exacerbating effects of any drug sensitivity or toxicity.

Two primary ways to promote steady blood drug levels are to choose, design or modify drugs to get satisfactory clearance and/or use special formulations to control how the drug is absorbed.

### Pharmacokinetics

Drugs in the body are eliminated by various mechanisms, including renal excretion, metabolism in organs (often the liver) and other transformations. Uptake has a separate set of dynamics, which for oral drugs can influenced by stomach pH, presence of food or gastric activity, and many more factors. Topical, sublingual, IP, IV and other modes all have relevant characteristics and sensitivities.

Some drugs accumulate in the body, particularly drugs that are compatible with fat (lipophilic). Some drugs are metabolized in minutes (insulin). Some are excreted rapidly. Nikkomycin Z (“nikZ”) (FIG. 1) is hydrophilic, eliminated mostly by renal excretion, with an elimination half-life of about 2.5 hours in humans.

For oral dosing, to get drug into the bloodstream typically requires the drug to pass into the GI tract, then into the blood. There is an array of mechanisms and characteristics of drugs, excipients, formulation and packaging used to improve uptake. For many drugs, uptake has varying degrees of efficiency. For nikZ, uptake from an oral dosage form may be on the order of 15-25%. When giving larger doses of nikZ, the efficiency of oral uptake decreases, decreasing slightly for single doses above about 375 mg to about 60% efficiency (7-12% net) for a single dose of 1000 mg, and even less efficient at higher single doses. Taking doses generally no larger than 500 mg at a time will maximize uptake. Dose ingestion repeated frequently can keep blood drug levels up. For nikZ, it appears that more than 50% of the oral drug may not add therapeutic value when taken only every 12 hours (“BID”), and perhaps even every 8 hours (“TID”, q. 8 hours).

Traditional ways of providing drug supply to offset short half-life elimination include continuous intravenous (IV) infusion. This is routine during hospitalizations and increasingly available on an outpatient basis, such as by using a “PortaCath” or similar devices. For chronic therapy, IV is less attractive when a suitable oral dosing form is available. Chronic therapy may require months and even years of dosing. For many reasons, patients prefer a tablet taken on a regular basis, such as daily (choose your hour), weekly (choose your day) or monthly (choose your date). Patterns make a regular dosing schedule easier to maintain.

Drugs can be formulated for extended release (XR), which can be particularly useful for short half-life drugs. Television advertisements for such formulations speak to the value of these in many applications. Developing such a formulation is often straightforward, but expensive and time consuming. Many vendors offer such formulation services. This is not a good fit for screening a drug that may or may not give a clinical benefit that might not warrant the effort of developing an XR formulation. Such formulations tend to be species sensitive, so even with a human formulation there is little value in testing that in rodents and perhaps even in dogs or other species. A formulation for mice likely would not be interesting for humans.

It is desirable to present drug in a way that can lead to blood drug levels useful for a therapeutic benefit, and helpful if the drug levels do not need to be dramatically higher than that therapeutic threshold, potentially wasting drug.

If an extended release can be simulated inexpensively, this can support testing and modelling how an XR formulation might be advantageous, allowing for evaluation of clinical results to better understand whether the effort of preparing an XR oral dosage form may be warranted. Formulation for extended release is well studied and well established, so the probability of developing such for nikZ and for many other compounds is high, available from multiple vendors.’

From experience in manufacturing development, stability of nikZ in aqueous solution at various pH and temperature conditions was known to be fairly short, only hours at pH 7 and room temperature, and worse at higher pH or higher temperature. It was not recognized until this invention that stability at about pH 3-3.5 at room temperature was both useful and practical for extended duration dosing of mammals.

### Chitin Inhibitors, Nikkomycin Z

Studying Hector et al. 1998 U.S. Pat. No. 5,789,387 shows the advantages of consistent and near continuous nikZ dosing. The present invention was prompted by a search for a simple system to facilitate testing of nikZ against various pathogens in mice. Studying nikZ closely, there appear to be many opportunities to treat a variety of pathogens. The present invention focuses on a simple system to facilitate testing of nikZ against various pathogens in mice. Briefly from that Hector 1998 patent:

Nikkomycin Z resembles N-acetyl glucosamine, the typical substrate for chitin synthase. NikZ inhibits various chitin synthase enzymes, resulting in reduced chitin content in the fungal cell wall protective structure. This in turn makes the cell less stable and susceptible to destruction by natural physiological processes.

Inhibitors of chitin synthesis are markedly effective in preventing morbidity and mortality in animals suffering from fungal infections, particularly when the inhibitor is administered continuously or in some approximation of continuity. In particular, nikkomycin Z has been demonstrated to be effective against a range of pathogens. In marked contrast to previous reports of use of nikkomycin Z, the duration of the new semi-continuous therapy is short, yet the results are superior to those of the prior art drugs. Hector 1998, U.S. Pat. No. 5,789,387, reported successfully using low doses of nikZ when using a continuous IV infusion (such as 33 mg/kg/day, rat) to enable 100% survival after a fatal IV challenge with the virulent B311 strain of Candida albicans. See FIG. 2A.

With oral absorption efficiency in many species, including human, reported to be about 10-15%, oral methods need to administer roughly ten times this amount, treating Candida with 200-300 mg/kg/day (rat, twice this for mouse, about 25% or 50-75 mg/kg/day in humans), or perhaps higher. Dosing for Coccidioides spp. is 25-75% of this level (2.5-30 mg/kg/day in humans, ˜200-2000 mg/day for a 70 kg human), preferably 400-1000 mg/day for a 70 kg human. Studies briefly reported here suggest oral availability may be more than 50% with frequent, small dosing.

Single doses above 375 mg (human, about 6 mg/kg) are less efficiently absorbed into the blood, with uptake nearly linear at 500 mg, notably reduced at 1000 mg, with significant saturation of uptake for single doses above about 2000 mg (human, about 33.3 mg/kg/dose). A clearance PK half-life of 2.5 hours works out to needing humans to take 250 mg BID (every 12 hours), and likely more.

In considering possible therapeutic models of interest to test an improved manufacture of nikZ and in particular noting that the literature has suggested benefits for higher frequency dosing, this invention was conceived, and tested directly. A notable feature of this invention is that animal handling is reduced to almost nothing outside normal care, requiring no oral gavage or injections. Such routes of administration become particularly tedious to the subject and the handler when dosing more than q. 12 hours and even more so for therapies of more than a few days.

### SUMMARY OF THE INVENTION

The present invention was conceived and demonstrated to provide a superior spreading of dosing episodes, more frequent than QID (q. 6 hours), spreading a daily dose over many hours and many intake events. This provides an imperfect but inexpensive presentation that will give useful information about the potential benefits of an extended release (XR) formulation. Drug-in-water (“DiW”), dissolving the test drug in water, is very convenient to manage in important animal models. Humans can follow the teachings of this invention and demonstrate again inexpensively a trial model and the potential value of preparing an XR formulation.

In one embodiment, the nikkomycin is nikkomycin Z administered in a semi-continuous fashion in an amount sufficient to treat infection in a mammal. In another embodiment, the amount of nikkomycin is sufficient to inhibit the enzyme chitin synthase for a period of time sufficient to result in reduced function of fungi, including killing fungi under some conditions. According to the present invention, continuous treatment with nikkomycin is particularly useful against systemic Coccidioides infections in humans. Additionally, semi-continuous treatment with nikkomycin is useful against more localized Coccidioides infections in humans.

The present invention also encompasses sustained release formulations of chitin synthesis inhibitors, including but not limited to a nikkomycin such as nikkomycin Z. In addition, the present invention encompasses intravenous administration, particularly a continuous infusion for multiple days. This is fairly common in modern therapy using a “porta-cath” on an inpatient or outpatient basis.

Treating disseminated coccidioidomycosis in mice with nikZ has not been previously reported. That the drug was effective is unsurprising, given many reports of nikZ efficacy in other animal models. Given the high safety of nikZ, high doses known to have no safety concerns were included, in combination with a novel route of administration, seeking in this study to overwhelm the disease. Not anticipated, beyond a general hope of some increased benefit, was the significant degree of benefit. This was dramatically successful.

What was previously unknown and untested was the new method of drug presentation, the center of this invention, and the ease of presentation to the subject. When the clinical benefit proved quite successful, this study looked at some of the dosing variables to better understand the details and potential. In “Study 2”, a much lower dose range showed benefit generally better than historical studies at the same dose levels. Further modeling, summarized here, suggests that drug utilization can be improved by a factor of more than 2 with this microdosing, compared to BID dosing. A BID dose of 50 mg/kg (100 mg/kg/day) may give an effect roughly equivalent to DiW microdosing with just 50 mg/kg/day (half the dose), or perhaps a still lower DiW dose. The benefit may be even greater. A future extended-release (XR) formulation may permit using still lower daily doses to achieve the same clinical effect, possibly ≤⅓ of the dose needed when dosing BID. Studies are continuing.

The present invention is directed to a method of formulating nikkomycin Z as a drug in water (“DiW”) and simply presenting that to a test animal as the sole source of drinking water. This is well accepted by several species. For drug of recent manufacture, the taste (to humans) is quite moderate and not unpleasant even at very high concentrations. Mice have consumed DiW solutions at up to high doses for up to 14 days with little or no hesitation. A cat accepted dosing in milk (single dose). The natural pH of a solution of the HCl salt of nikZ is about pH 3.3 (3.2-3.5 is common). NikZ in solution at 0.5-5 g/L degrades about 1% per month refrigerated, and about 1% per day at 25° C., conveniently only about 4% after 4 days. This is convenient for a test animal water bottle change over even long, holiday weekends. Water can be changed at any higher frequency fitting local protocols. The main degradation products of nikZ under these conditions are well characterized and non-toxic. No toxicity concerns have been identified for nikZ degradation products at the levels seen.

About pH 3.3 is conveniently a pH commonly used for water supplied to laboratory mice (pH 2.8-3.3) under good laboratory practices. This is also in the general pH range common for wines (3.0-3.4 for white wines, 3.3 to 3.6 for reds) and many foods. Cranberry juice has a pH of approximately 2.3 to 2.5.

Mice likely are not drinking consistently at an even pace through their waking periods. The model shows that dosing even every two hours gives a fairly narrow range of blood drug levels, within about 3% of C_{average} (Cₘᵢₙ of 97 or 98% of C_{avg}). The clinical effect in mice showed that whatever was their actual pattern, it was enough to dramatically reduce the infection challenge. Two PK studies subsequent to August 2020 showed a wide spread in rat blood levels, consistent with the general models detailed here. At some times of day, levels were mostly above the 170 ng/mL of the Hector 1998 patent. At other times of day the levels were much lower. As expected, natural drinking gives a significant spread of blood drug levels.

Studies are continuing, seeking to more precisely understand what is a minimal effective dose, and other dosing parameters that may make still lower doses practical and effective. There are many reports of nikZ studies in a variety of species (mice, rats, dogs, humans, more) and routes of administration (oral QD, BID, TID, QID, IV, IP, SC, various sustained release formulations).

The important thing for therapeutic effect is the blood drug levels—concentration, time at concentration, and consistency of concentration (if only modestly above the effective therapy blood drug level). If blood drug levels can be tolerated at levels significantly above the effective therapy blood drug level (such as 125%, 150%, 200% of effective therapy blood drug level, and even higher), wider variations in momentary blood drug levels will still keep the blood drug levels mostly or completely above the effective therapy blood drug level. The significant safety of nikZ permits considering such levels. This is discussed in more detail below. More studies will help better understand which changes can be tolerated without reducing clinical benefit.

In July 2021, two articles were released detailing the studies mentioned in this patent, and more. These articles are incorporated here by reference and fully incorporated.
Sass G, Larwood D J, Martinez M, Chatterjee P, Xavier M O, Stevens D A. NIKKOMYCIN Z AGAINST DISSEMINATED COCCIDIOIDOMYCOSIS IN A MURINE MODEL OF SUSTAINED RELEASE DOSING. Antimicrob Agents Chemother. 2021 July 12:AAC0028521. doi: 10.1128/AAC.00285-21. Epub ahead of print. PMID: 34252303.
Sass G, Larwood D J, Martinez M, Shrestha P, Stevens D A. Efficacy of nikkomycin Z in murine CNS coccidioidomycosis: modelling sustained-release dosing. J Antimicrob Chemother. 2021 July 16:dkab223. doi: 10.1093/jac/dkab223. Epub ahead of print. PMID: 34269392.

### BRIEF DESCRIPTION OF THE FIGURES

FIG. 1 shows the chemical structure of nikkomycin Z.

FIG. 2A shows plasma concentration of nikkomycin Z following continuous intravenous administration of nikkomycin Z to a rat. (Hector 1998 U.S. Pat. No. 5,789,387, FIG. 5) A loading dose of 40 mg/kg was infused over a period of 2 hours (20 mg/kg/hr, 480 mg/kg/day). A maintenance of dose of 33 mg/kg/day (1.38 mg/kg/hr) was then infused to achieve a steady state concentration of about 170 ng/ml.

FIG. 2B shows plasma concentration after single doses in a human, single ascending dose study. The table on the left includes the smallest dose tested, 250 mg as a single dose. Nix 2009, Nix, D. E.; Swezey, R. R.; Hector, R.; Galgiani, J. N. Antimicrob. Agents Chemother. 2009, 53, 2517. That 250 mg curve is repeated in FIGS. 8B and 8C.

FIGS. 3A-E: Show disease burden in lung, liver, spleen after IV infection, inducing disseminated coccidioidomycosis, then dosing with nikZ DiW or IP intraperitoneal BID at the same daily dose levels. n=10 in all groups. (Study 1 3A-C, Study 2 3D-E)

FIG. 3A. Fungal loads in lungs: t-Test: *p≤0.05, ***p≤0.001. Comparisons without bracket for each group: water vs all other bars. Other comparisons as indicated by the ends of the bracket. FIG. 3B. Fungal loads in livers: t-Test: **p≤0.01, ***p≤0.001. Comparisons without bracket for each group: water vs all other bars. Other comparisons as indicated by the ends of the bracket. FIG. 3C. Fungal loads in spleens: t-Test: ***p≤0.001. Comparisons without bracket for each group: water vs all other bars.

FIG. 3D shows dose dependent disease reduction in Study 2, a second animal study, with a higher infection challenge. FIG. 3E shows a survival curve from Study 2.

FIG. 4 charts mouse drinking in Study 1. The daily measurement data shows overall drinking patterns but lacks detailed information over hours or minutes. More detail will be sought in future studies. The dosing was sufficient for clinical benefit, despite details of uptake variation and uncertainty.

FIG. 5A shows blood drug levels in a model of microdosing steadily at q=30 minutes (every 30 minutes) over 96 hours, starting at 6 am on day 1. This also models one representative model with a 10% randomization factor at each 30 minute time slice (offset+0.05 μg/ml higher for clarity), and a second representative model with a 20% randomization factor (offset+0.1 μg/ml for clarity). FIG. 5B illustrates the effects of dosing frequency. Comparing the essentially flat line when dosing at 30-minute intervals (FIG. 5A, without randomization), FIG. 5B shows a model of steady dosing at 2 hour intervals (solid line), 4 hour intervals (dashed line), and 8 hour intervals (dotted line).

FIG. 6A show a model including a sleep interval of 8 hours, plus a “sleep+4” four hours into the sleep cycle. FIG. 6A shows this pattern with no boost (solid line) and an “8×” boost (8 units in addition to 32 units during waking hours). FIG. 6B shows a “2×” boost (2 at 2 am+32 during waking). FIG. 6C shows a “4×” boost. FIG. 6D shows “10×” (solid line) and “12×” (dashed line) boosts.

FIGS. 7A and B shows the impact and benefit of a variety of loading dose patterns, built around dosing every two hours during a waking period.

FIG. 8A illustrates another pattern working in many elements from this invention. This example is based on 200 mg per day, in a pattern that gives blood drug levels almost always above 62.5 ng/mL, a level expected to be sufficient to kill the VF fungus.

FIG. 8B illustrates this dosing pattern scaled to 1200 mg/day (dashed line) and 2400 mg/day (solid line).

FIG. 8C is an expansion of part of FIG. 8B, at 1200 mg/day (human daily dose), focusing on the first 40 hours. This is shown as a dashed line, centered on the (1200) line at about 0.6 μg/mL. FIG. 8C includes a modeled blood drug level curve without loading dose modifications (thin solid line), which also reaches a steady state C_{average} at about the same 0.6 μg/mL.

FIGS. 9A-9E illustrate a dosing vessel, basically a graduated cylinder configured to facilitate easy drinking in small portions, facilitating tracking of the momentary and overall extent of consumption.

FIGS. 10A-10D illustrate various parameters potentially important to designing a therapeutic regimen.

### DETAILED DESCRIPTION OF THE INVENTION

The present invention is directed at in improved therapy for a disease of particular concern and interest. The present invention also is directed at an improved dosing method, particularly to facilitate pre-clinical efficacy testing, but also well suited to human therapy, particularly in early dosing evaluation.

The present invention provides methods of preventing morbidity and mortality in mammals due to fungal infections by the semi-continuous or continuous administration of chitin synthesis inhibitors.

The improved dosing model is directed to drugs with a relatively high clearance rate, such as a half-life less than about 6 hours, making dosing more frequently than every 12 hours important for optimizing pharmacokinetics and reasonably stable blood drug levels. The improved dosing model works particularly well for drugs that are soluble in water, and sufficiently stable to provide a useful dose for at least the preferred daily dosing duration.

The present invention was conceived and demonstrated to provide a superior spreading of dosing episodes, more frequent than QID (q. 6 hours), spreading a daily dose over many hours and many intake events. This provides an imperfect but inexpensive presentation that will give useful information about the potential benefits of an extended release (XR) formulation. Drug-in-water (“DiW”) is very convenient to manage in important animal models. Humans can follow the teachings of this invention by drinking a daily dose of drug in water over the course of a day, with attention to the rate of consumption. Modeling suggests that ingesting a unit dose in each time period T with relative consistency during waking hours, where the unit dose is the daily dose divided by the fraction of T per 24 hours, and where T is less than about 150% of the clearance half-life of the drug, will maintain blood drug levels to within about ±15% of C_{average}, the average blood drug concentration. Maintaining consistency within about 80% of the clearance half-life will maintain blood levels within a tighter tolerance, for example <±3% keeping dosing on a regular q. 2-hour dose for a drug with a clearance half-life of 2.5 hours for small doses.

This DiW method was intended as an interim test administration, as a possible suggestion of the potential value of preparing an XR formulation. The DiW method proved easy to administer, and to have greater than anticipated clinical benefit.

According to the methods of the present invention, nikkomycins have been found effective in treating a mammal having an infection due to Coccidioides spp. Such fungi include C. posadasii, and C. immitis. From tests against and reports on other dimorphic fungi, many of these too will respond, at a blood drug level appropriate to each fungus. The semi-continuous DiW microdosing method makes this more practical, easy to administer, giving stable to very stable blood drug levels.

In one embodiment, the nikkomycin is nikkomycin Z administered in a semi-continuous fashion in an amount sufficient to treat infection in a mammal. In another embodiment, the amount of nikkomycin is sufficient to inhibit the enzyme chitin synthase for a period of time sufficient to result in the death of the fungi. According to the present invention, continuous treatment with nikkomycin is particularly useful against systemic Coccidioides infections in humans. Additionally, semi-continuous treatment with nikkomycin is useful against more localized Coccidioides infections in humans.

The present invention also encompasses sustained release formulations of chitin synthesis inhibitors, including but not limited to a nikkomycin such as nikkomycin Z. In addition, the present invention encompasses intravenous administration, particularly a continuous infusion for multiple days. This is fairly common in modern therapy using a “porta-cath” on an inpatient or outpatient basis.

Treating disseminated coccidioidomycosis in mice with nikZ has not been previously reported. That the drug was effective is unsurprising, given many reports of nikZ efficacy in other animal models. Given the high safety of nikZ, high doses known to have no safety concerns were included, in combination with a novel route of administration, seeking in this study to overwhelm the disease. Not anticipated, beyond a general hope of some increased benefit, was the significant degree of benefit. This was dramatically successful.

What was previously unknown and untested was the new method of drug presentation, the center of this invention, and the ease of presentation to the subject. When the clinical benefit proved quite successful, this study looked at some of the dosing variables to better understand the details and potential. In “Study 2”, a much lower dose range showed benefit generally better than historical studies at those dose levels. Further modeling, summarized here, suggests that drug utilization can be improved by a factor of more than 2 with this microdosing, and suggests that a full XR formulation may provide a benefit of about 3 and perhaps more. That is to say a dose given twice a day if divided into a microdose presentation can give the same effect at half the daily drug delivered, a BID dose of 50 mg/kg (100 mg/kg/day) gives an effect roughly equivalent to microdosing with just 50 mg/kg/day. The benefit may be even greater. Studies are continuing.

An additional benefit is the potential for clinical evaluation heretofor difficult to study. XR formulations with zero order release profiles generally support maintaining very steady blood drug levels. Given the realities of commercial manufacturing, dosage units are generally chosen, and changing a dose presentation by 5%, or 1% is simply not practical. Such fine grained changes can be practical with the XR formulation. An XR formulation is fine grained, such as coating particles of drug substance to give microbeads of XR release drug product, and metering the dose of such microbeads. This is not generally done, with no known, reported studies that have attempted this. A typical dosing protocol is to overwhelm the disease, so finding minimum effective dose thresholds may be less interesting for clinical application.

The teachings of this invention can be readily modified to refine dosing with high precision, allowing study of the effect of a very small change in blood drug level, such as 5%, and perhaps less, which may help provide information on just what levels impact a pathogen in a given patient, which then opens the possibility of studying the clinical effect between patients (with varying comorbidities and physiologies). A general expectation is that this is not clinically relevant, but that to some degree is driven by not having such a tool. The possible studies may be interesting or not, but this will at least facilitate the study.

Studying clinical effects of a somewhat steady dose level can inform what may be likely targets for clinical testing formulations for humans. The studies as conducted showed a clinical benefit superior to treating with oral gavage every 12 or 8 hours, so the blood drug levels are clinically relevant. Current studies of more detailed PK in rats and mice prove to be complex, but generally consistent with the observed clinical benefit.

Further, microdose delivery reduces Cₘₐₓ (maximum blood drug level concentration) when compared to a bolus of the same daily total. An example of dividing is to divide the daily dose by four and give the divided dose four times a day, nominally evenly spaced at every 6 hours. Dividing a daily dose into three equal portions requires each dose to be larger than when divided by 4, with still higher Cₘₐₓ. Dividing by two gives a still higher unit dose and Cₘₐₓ. Limiting Cₘₐₓ is generally expected to improve safety. Yet another benefit is that smaller episodic doses are less likely to exceed limitations on oral uptake. Together, this means that larger daily total doses can be delivered more effectively, which presents the possibility of treating more challenging infections.

More generally, this invention is directed to presenting any drug in a suitable dietary component, including food, drinking water, or solution form. This is particularly helpful for a drug with rapid elimination. This presumes that the drug can be sufficiently stable under the test conditions. Drinking is naturally intermittent, as is eating. These frequencies will be discussed in more detail.

Nation 2016 reports chronic administration of CNO through drinking tubes (see FIG. 4 of Nation, for example). H L Nation et al, DREADD-induced activation of subfornical organ neurons stimulates thirst and salt appetite, J. Neurophysiology 115: 3123-3129 (2016). During therapy, exciting SFO neurons increased daily water intake from about 2 ml/day (with or without CNO) to about 4 for the duration of therapy, with a clearance half-life of about 1 day.

Reviewing basic pharmacokinetics, it is evident that a controlled oral dosing format can make more efficient use of drug. In a human study (Nix 2009), a single dose of 250 mg had an AUC of 11. See FIG. 2B. A human dose of 500 mg/day (250 mg BID) has been expected to provide some therapeutic benefit, particularly for milder cases. This would project to an AUC of 22. Shubitz 2014 estimates that with BID dosing an AUC of about 33 should confer ED80 level protection, although human 250 mg BID was shown at 35, not 22.

Using the blood drug levels reported in Nix (human PK curve, ng/ml over time after dosing), in a model dividing that dose very finely to provide about 2% every 30 minutes (actually 1/48^{th}) and summing the effect of sequential, overlapping doses, gives a modeled AUC of 2.9, and a blood drug level of 122 ng/ml continuously. Modeling dose slices of every 30 minutes gave a very even projected blood drug level. Dosing in units of every 2 hours still gave fairly tight spreads of the mean of 240 ng/ml±6%. Dosing every 8 hours gave a much wider spread of 91%, 92-426 around a mean of 259 ng/ml, AUC 5.80 for all dosing patterns. Dosing every 4 hours gave a spread of 22%. Taking drug every 4 hours or less provides superior range in dose level swings.

The Nix study led to a recommendation of 250 mg BID as a possibly effective low dose. Modeling microdosing at 500 mg/day gives a continuous blood drug level of 244 mg, a level seen at about 11 hours after a single tablet of 250 mg. In a preferred embodiment, consider 600 mg/day a convenient human dose (600 divides nicely to 50 mg/2 hour segments, a preferred embodiment of this invention). In a 65 kg human, this is 9.2 mg/kg/day, and corresponds roughly to 78 mg/kg/day in a mouse. In the most severe disease challenge test in mice, this dose level gave about 50% reduction in disseminated disease after only 5 days of therapy.

This finely divided dose format gives an AUC of about 25% of the single dose, saving considerable drug if indeed this is a therapeutic dose.

Testing a variant of this for mice proved fairly simple, once conceived, in dissolving drug in drinking water (DiW) and providing that to mice as their sole water supply. The mice readily accepted rather high doses. Mice challenged with about 200 arthroconidia by IV injection were almost completely protected from residual infection after treating with nikZ at levels of 200 mg/kg/day (and higher). Mice injected with an extremely high challenge of about 1000 arthroconidia in the same system showed therapeutic benefit from even 20 mg/kg/day nikZ, and some 75% reduction of disease when treating at 150 mg/kg/day, 90% at 400 mg/kg/day. Longer duration therapies will be studied.

These doses suggest a human level of 1200-2000 mg/day may be required, AUC 14-22, a reasonable alignment with the Nix AUC of 22 (AUC=11 for 250 mg, ×2 BID). The therapeutic dose may get lower with more control of dosing rate than can be achieved with DiW in mice.

Mice likely are not drinking with high repeatability in drink size or frequency through their waking periods. The model projects that dosing even every two hours gives a fairly narrow range of blood drug levels. The clinical effect in mice showed that whatever was their actual pattern, it was enough to dramatically reduce the infection challenge.

Studies are continuing, the better to more precisely understand what is a minimal effective dose, and other dosing parameters that may make still lower doses practical and effective. There are many reports of nikZ studies in a variety of species (mice, rats, dogs, humans, more) and routes of administration (oral QD, BID, TID, QID, IV, IP, SC, various sustained release formulations). The important thing for therapeutic effect is the blood drug levels—concentration, time at concentration, and consistency of concentration (if only modestly above the effective therapy blood drug level). If blood drug levels can be tolerated at levels significantly above the effective therapy blood drug level (such as 125%, 150%, 200% of effective therapy blood drug level, and even higher), wider variations in momentary blood drug levels will still keep the blood drug levels mostly or completely above the effective therapy blood drug level. NikZ safety makes such levels worthy of consideration. This is discussed in more detail below. More studies will help better understand which changes can be tolerated without reducing clinical benefit.

### IV First Order

As this invention draws on Hector 1998, that patent is quoted heavily here. Resources are not yet available to provide a controlled, continuous drug delivery by IV infusion. Mice do not handle IV infusions well. Rats require larger drug supply, and the compound is still limited in supply. Vendors are readily able to run such tests when more drug is available. Preparing extended release oral dosing forms is expensive and time consuming. These also are species specific, suggesting a focus on human-only XR development.

This provides a simple model and process for animal dosing. The results reinforce the anticipated benefits from human IV therapy. This also suggests value in developing an extended release (XR) formulation for oral dosing. It is likely that particularly severe disease will be well treated by IV infusion, even on an outpatient basis. Milder forms of disease are likely to respond to oral dosing. Oral dosing also is useful as a follow-on after IV therapy. It has been observed that some fungi may respond only to higher doses of nikZ. Here again the IV format may prove advantageous.

### Oral Zero-Order Microdose

This invention takes advantage of the historical demonstration of a benefit in administering more frequent, smaller doses to improve nikZ uptake and to improve consistency of blood drug levels throughout the therapy. (Hector 1998 patent, C. albicans). In the limit, a continuous IV infusion may be ideal, or an XR oral dose. Neither are practical to test in mice, leading to the inventive idea of a semi-continuous, very convenient simulation by providing drug in drinking water (“DiW”), anticipating some episodic intake that would sufficiently average out to provide satisfactory blood drug levels over each 24-hour period and to effectively treat the disease challenge.

Working from the general idea in Hector 1998, drug in water (DiW) was presented as the drinking water for mice, at a cage level in groups of 5. Various concentrations were presented to give different doses. Nikkomycin Z is nearly tasteless, with basically a mild umami or citrate taste. The mice accepted the DiW with no hesitation at very high dose rates (10 g/L, or 50 mM of free base, as the HCl salt). Drug in this form is stable in the water bottles for several days, so presenting a dose for 3 or 4 days of water supply is effective for drug supply. Typical good animal practices call for cleaning cages 2 or 3 times a week, a convenient time to present a fresh DiW supply. Unused drug can be recovered from the solution by simple chemical processing. It is more efficient to prepare a minimal excess of drug, since maintaining the dry powder is highly preferred to handling and storing various solutions or undertaking recovery efforts.

This new DiW invention is quite easy to practice. From the start, note that whatever may be the variables and variability, the result was highly effective. Although drinking is necessarily neither continuous or rigidly precise in rate of drinking, this method still divides the dosage increments into many dose increments, according to whatever size of drinks or gulps the mice take. This mode means that a sleeping animal will not be taking in drug. Some pharmacokinetic (PK) modeling is discussed later.

Note that a high variation in blood drug levels that nevertheless keeps the concentration over time high enough to provide a therapeutic benefit provides some value in exploring dosing alternatives. As discussed in more detail below, working with a more cooperative mammal such as a human, a patient can be asked to follow a protocol to provide more precise control of blood drug levels. This will allow for closer study of just what blood drug levels are critical.

There are several ways to do this. One preferred method is to provide dosing units that a patient can take easily on a schedule they can manage. Dosing every 2 hours is very effective in flattening the variation in blood drug levels. Dosing every 4 hours is much better than dosing every 6 or 8 hours, for flattening the variation.

Even better than two-hour doses is to dissolve a period dose in water and drink the dose at a rate proportional to the period. A human patient is instructed to consume DiW at a constant rate during waking hours.

This is easy to do by providing a periodic dose in a container marked with a dosing schedule. Either from clock time or relative hours, the human drinks to each hour line roughly by each clock hour. Since the blood drug level is the result of a number of small doses, each with an independent absorption curve, moderate and even fairly significant deviance from this “drink one unit per time period (30 minutes)” does not have a large impact on the approximately steady state level of drug in the blood.

In another preferred implementation, doses are provided in divisible increments. It is convenient to provide a unit dose sized to take every 2 hours. For “TID” dosing (three times a day, every 8 hours), this would include 4 such 2-hour unit doses. A tablet or capsule is easy to handle and to take. The patient can take the 8-hour grouping at a single time (gives every 8 hour dosing), one unit every 2 hours (gives good smoothing in blood drug levels), or any combination that the patient chooses to follow. Taking drug in 8-hour time units gives wide swings in blood drug levels. Taking drug in 4-hour increments gives smaller swings, and 2-hour increments gives fairly small swings.

For a willing patient, consuming drug in even smaller increments further reduces swings. It appears the drug is more effective when swings are smaller. Dosing three times a day seems to be significantly more effective than two times a day. Running trials may help to understand if more finely divided dosing confers clinical benefit that outweighs the inconvenience of higher frequency dosing.

This “dose spreading” can be further improved by providing the tablet in a form that dissolves well in water. In one preferred implementation, the patient dissolves an 8-hour grouping in some quantity of water, for example in 8 ounces (or 80 milliliters, or other convenient volume). Cough syrup is often provided with a 20 milliliter dosing cup, recommending “fill to 20 ml, consume at the dosing schedule.” The same works well here, drinking for example 1 of 8 ounces (29.6 or ˜30 ml) per hour. The dosing model works even better if the patient takes a 30 minute dose unit approximately every 30 minutes. A bottle holding 8 ounces is easy to carry around. The drug is stable enough that a patient can make up three such 8 hour bottles at one time and keep them accessible as needed during the day.

Stopping dosing, specifically when sleeping causes a significant drop in blood drug levels fairly quickly. The effect would be the same if delaying dosing while watching a movie or sporting event, although taking a 2-hour pill on schedule could be fairly convenient during such waking activities. In the PK modeling described below, see the notes that a special dose about four hours into a sleep period will mitigate this drop. According to the model, the “Sleep+4” special dose can be 2 to 8 times the size of a typical half-hourly dose unit. A range of patterns is likely to give acceptable therapeutic effects. The patient could drink 2 or 3 “hour units” just at bedtime, raising the basic level, and limiting the minimum concentration. Similarly, a large consumption upon waking can help restore levels. The smoothest overall curve allows for the lowest effective dose and clinical benefit, as this keeps the blood drug level consistent (discussed in Hector 1998), allowing utilization of somewhat lower doses. This is basic pharmacokinetics, as taught in the UCSF Pharmaceutical Chemistry PhD program in 1979, and many places before and since. Other dosing concepts here also are well known to one skilled in the art.

The novelty here is that the DiW method works, and works well enough despite a drop in drug levels during sleep periods. A formal PK study is underway. The blood drug levels reached in Study 1 reported here proved to have significant, even dramatic antifungal effect.

In this model, the 200 mg/kg/day dose (1 g/L, 2 millimolar DiW) should give drug levels very close to 0.1 μg/ml in the blood—IF—the administration is highly regular (no sleep periods). Adding 8 hours of sleep and the “sleep+4” dose of 8 times the normal dose (normal meaning the 30-minute dose aliquot, so 4 hours' worth of dose) gives swings of about 20% above and below the nominal steady state level, 80-120 ng/ml. Using no extra dosing during an 8-hour sleep cycle takes the blood drug level to 20 ng/mL, then resuming pro-rata consumption for 16 hours takes the drug levels to 145 ng/mL. The “sleep+4” 8× cycle reaches a lower maximum because the boost dose of about 17% ( 4/24) of the daily aliquot means the remaining 83% of the drug is consumed during the waking period at 17% smaller pro-rata units than if the waking period of 16 hours needs to cover 100% of the drug.

With interspecies allometric scaling, this suggests that a human dose of about 150 mg by IV over 24 hours may provide significant clinical benefit. The DiW microdose level should be on the order of 1 to 1.7 grams

The DiW method has significant swings in blood drug level, highly dependent on dosing rate (rate of sipping). This is still useful for a screening tool. For humans, it can be quite helpful for clinical trials, to establish basic dosing parameters, significantly including both dose level, and also dose duration (days of therapy by this method). Humans can choose to regulate their intake both closely and with high regularity, particular if the therapy period is less then 12-21 days. Humans with the most serious disease, who may very well respond to this therapy, will be highly motivated to follow the schedule.

By semi-continuous administration is meant administration at a more or less consistent rate for periods of time, preferably more than 8 hours and more preferably about 16 hours, with some intervening periods of no dosing (periods of sleep, or inattention to dosing). In general, semi-continuous administration approaches continuous administration so the discussion of either is useful in considering the perspectives of the administration.

By continuous administration is meant administration in which a more or less constant amount of nikkomycin or other chitin synthesis inhibitor is delivered per unit time to the subject mammal and in which a more or less steady state concentration of the nikkomycin or other chitin synthesis inhibitor is achieved in the plasma or in a target organ of the subject mammal. This is to be contrasted with discontinuous methods of administration in which the nikkomycin or other chitin synthase inhibitor is delivered either once or repeatedly, with large amounts of time separating each repeated delivery. It is believed that continuous administration results in the achievement of prolonged and consistently high levels of nikkomycin or other chitin synthesis inhibitors in the plasma or target organs of the subject mammal. In contrast, discontinuous administration is believed to result in the achievement of an initial high level of nikkomycin or other chitin synthesis inhibitor followed by a fall in levels until the next delivery again results in a high level.

### Dose

The Nix 2009 data lists the human blood drug level at 12 hours (time for the next BID dose) as 200 ng/ml, about three times a sometimes cited anticipated therapeutic level of 65 ng/ml. Modeling the DiW system, a dose of 500 mg/day should give a mean blood drug level of 245 ng/ml, 7.7 mg/kd/day in a 65 kg human, with an AUC of 5.8 for 24 hours.

Note that the Hector 1998 maintenance blood drug level was about 170 ng/ml in rats with a continuous IV infusion of 33 mg/kg/day (1.4/hr), after a loading dose of about 14 times this for 2 hours (20/hr). Referring to FIG. 2A shows an early peak of about 5000 ng/ml (˜30×170 ng/ml, briefly). Based on reports and expectations that the MIC level against Coccidioides is about 63 ng/ml, this suggests that repeating the Hector method with Coccidioides would require about ⅓ of the Hector levels, or 0.37 mg/kg/hour for a blood drug level of 62 ng/ml (in rats), then add 10% for a safety margin to about 0.4 mg/kg/hr. Rat doses correlate to human doses at about a factor of about 4, suggesting 0.1 mg/kg/hr in humans (6.5 mg/hr, 156 mg/day). Considering that oral doses are estimated to be about 15-25% absorbed, 156 mg/day in the blood would call for dosing with about 625-1000 grams PO, very much in the anticipated therapeutic range.

If the anticipated therapeutic level is indeed 65 ng/ml, this models as requiring 130 mg for a total human dose. This is based on many assumptions all holding true, so discounting this to double that minimum brings the estimate back about 250 mg (per day, half the Nix 2009 low dose model). A human PK study will answer the uptake calibration. A phase 2 study in humans will give efficacy information (what blood drug levels are needed to address disease of a given severity).

Since nikZ is quite safe, an IV infusion rate of 0.77 mg/kg/hr (18.5 mg/kg (mpk), 1.2 g/day for a 65 kg human) should be very effective, giving average blood drug levels of about 125 ng/mL.

### Other Indications

The methods of the present invention are particularly suitable for the treatment of infections due to dimorphic fungi, including Coccidioides spp., Blastomyces spp., Histoplasma spp., Paracoccidioides spp. and Sporothrix spp.

C albicans

In particular, Hector's continuous administration of nikkomycin Z has been found to be especially effective in combatting infections due to Candida albicans. Although the prior art taught that nikkomycins were of limited effectiveness against Candida spp., Hector surprisingly found that when administered in a continuous fashion, nikkomycin Z is highly effective. DiW oral microdosing should be highly effective against Candida, albeit at a significant dose level. The relatively brief therapeutic duration offsets the tedium of DiW dosing.

The methods of the present invention are particularly suitable for the treatment of infections due to Candida spp. Such Candida spp. include, but are not limited to: Candida albicans, Candida parapsilosis, Candida krusei, Candida tropicalis, Candida glabrata.

### Broadening

In one embodiment of the present invention, the nikkomycin is administered in a continuous or semi-continuous fashion in an amount sufficient to treat infection of the fungus in a mammal. In another embodiment, the amount of nikkomycin is sufficient to inhibit the enzyme chitin synthase for a period of time sufficient to result in the death of the fungi.

As used herein, therapeutically effective means able to result in clinical improvement in the signs symptoms of disease and/or prevention of mortality in the more critically ill. Therapeutically effective amount or concentration means an amount or concentration sufficient to result in clinical improvement in the signs and symptoms of disease and/or prevention of mortality in the more critically ill. Minimum effective concentration (MEC) means the minimum concentration of a chitin inhibitor in plasma or a target organ (e.g., kidney) that is therapeutically effective.

The subject of the methods of the present invention is a mammal, including but not limited to mammals such as cows, pigs, horses, cats, dogs, etc., and is preferably a human. In the case of human subjects, the subject may be an immunocompromised subject such as one who suffers from acquired immune deficiency syndrome (AIDS), is neutropenic, or is undergoing immunosuppression for transplantation, therapy for cancer, or various current drugs, including some used for rheumatoid arthritis.

### Drug Families

In addition to nikkomycins, other chitin synthesis inhibitors will be suitable for use in the methods of the present invention. Examples of such chitin synthesis inhibitors are polyoxins, such as polyoxin D.

### DS=Drug Substance

NikZ drug substance is 95% pure according to current manufacturing (by hplc, UV detection of chromophores). With the HCl salt and some water of hydration, the activity is typically 75-82%. Mass balance, elemental analysis, and more lead us to conclude that nothing else is in the drug substance. Identity is confirmed by the characteristic UV spectrum eluting as expected on hplc, with more elaborate confirmation by NMR and IR as needed.

### Extended Discussion

### Routes of Administration and Sustained Release Compositions

The inhibitors of chitin synthesis employed in the methods of the present invention may be administered by any method such that at least a minimum effective concentration (MEC) of inhibitor is maintained in the plasma or in a preselected organ of the subject mammal for a time sufficient to be therapeutically effective.

In a preferred embodiment, the method of administration is by continuous intravenous delivery. Such continuous intravenous delivery is to be contrasted with discontinuous methods of delivery such as: the bolus intravenous injections of nikkomycin Z given once daily in Becker et al., J. Infect. Dis., 1988, 157:212-214; the oral administration of Hector and Schaller, Antimicrob. Agents Chemother., 1992, 36:1284-1289; and the subcutaneous injections of Chapman et al., 1993, Abstracts of the Conference on Candida and Candidiasis: Biology, Pathogenesis, and Management. Abstract No. A/20. It is believed that continuous intravenous delivery permits the buildup and maintenance of a therapeutically effective concentration of chitin inhibitor in the plasma or affected organs of the subject mammal.

In addition to continuous intravenous administration, other routes of continuous administration are useful in the present invention. For example, well known sustained release methods that permit sustained release of chitin synthesis inhibitors by the per-oral, intramuscular, or subcutaneous routes are effective as long as the sustained release method is effective in building up an MEC in the subject mammal for a time sufficient to be therapeutically effective. Sustained release methods include diffusion systems in which the rate of release of a chitin synthesis inhibitor is determined by its diffusion through a water-insoluble polymer. The inhibitor can be present as a core surrounded by the polymer, as in reservoir devices; alternatively, the inhibitor can be dispersed in a matrix of polymer. Other sustained release methods include the use of implants for subcutaneous tissues and various body cavities as well as the use of transdermal devices. For a discussion of sustained release methods see Longer, M. A. and Robinson, J. R., Chapter 91 in Remington's Pharmaceutical Sciences, Gennaro, A. R., ed., Mack Publishing Company, Easton, Pa., 1990.

Hector 1998 U.S. Pat. No. 5,789,387 details many aspects of formulation useful with nikZ. See Col. 5, line 57 through Col. 8, line 19, incorporated herein by reference.

### Pharmaceutically Acceptable Vehicles

U.S. Pat. No. 5,789,387: “Pharmaceutically acceptable vehicle means a carrier suitable for delivering safe and therapeutically effective amounts of the nikkomycin or other chitin synthesis inhibitor. Such a vehicle includes but is not limited to saline, buffered saline, dextrose, water, glycerol, ethanol, dimethyl sulfoxide, and combinations thereof. The vehicle and nikkomycin or other chitin synthesis inhibitor can be sterile. The vehicle should suit the mode of administration.

“In a preferred embodiment, the chitin synthesis inhibitor is formulated in accordance with routine procedures as a pharmaceutical composition adapted for intravenous administration to human beings. Typically, compositions for intravenous administration are solutions in sterile isotonic aqueous buffer. Where necessary, the composition may also include a local anesthetic such as lignocaine or lidocaine to ease pain at the site of the injection. Where the composition is to be administered by infusion, it can be dispensed with an infusion bottle containing sterile pharmaceutical grade water or saline.” (end of quote). This language is relevant in the present invention as well.

For oral uptake, such as by DiW, at the native nikZ HCl salt pH of about 3.35, the solution is stable for days. If mixed at physiological pH, such as by dilution in phosphate buffered saline, the stability is lower. One method of maintaining a highly stable IV formulation is to have a small container of nikZ at about pH 3.35, mixed shortly before infusion with a carrier such as in sterile isotonic aqueous buffer, D5W, normal saline, PBS, and the like. The concentrated source can be maintained at 20 g/L (up to saturation, about 25 g/L when chilled, such as if refrigeration is expected during distribution or handling). In one preferred embodiment, a dose of about 180 mg is administered to a human by IV infusion over 24 hours. At 10 g/L, this can be prepared in 18 mL. One way to deliver this is to add 3 ml of 20 g/L nikkomycin Z to 250-500 mL of normal saline and infuse this over 8 hours. Repeat for the duration of therapy.

### Nikkomycins

U.S. Pat. No. 5,789,387: “In a preferred embodiment of the present invention, the chitin synthesis inhibitor is a nikkomycin. Nikkomycins are a class of chitin synthesis inhibitors that are thought to act by inhibiting the enzyme chitin synthase (Cabib, E., Antimicrob. Agents Chemother., 1991, 35:170-173). Nikkomycins are peptidyl nucleoside antibiotics produced by Streptomyces (Isono et al., 1965, Agricultural Biology and Chemistry 29:848-854).

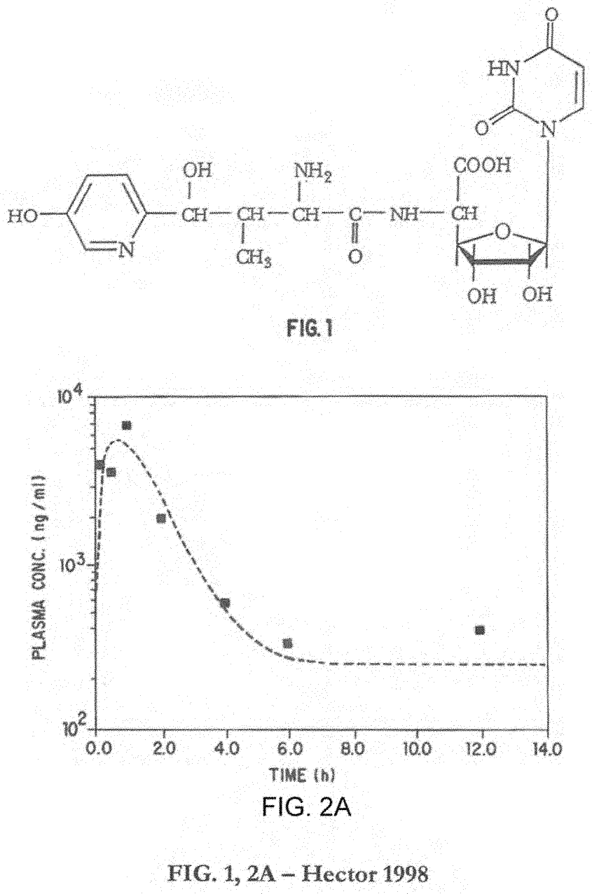

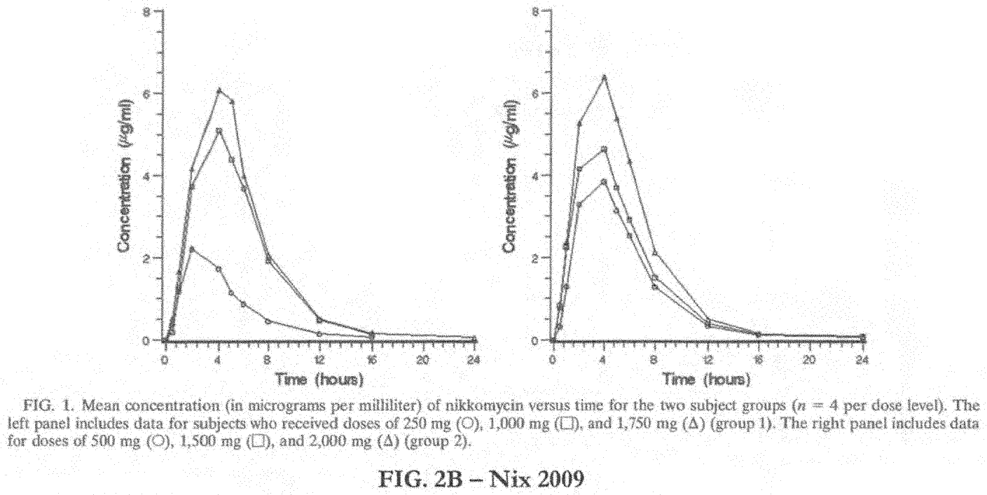

“One of the better studied nikkomycins is nikkomycin Z. See FIG. 1 for the chemical structure of nikkomycin Z. Due to its close structural resemblance to UDP-N-acetylglucosamine, the substrate of chitin synthase, nikkomycin Z is a potent inhibitor of this enzyme in fungi, insects, and arthropods (the only organisms containing chitin). The available evidence suggests that nikkomycin Z shows no toxicity against plants, fish, or mammals under reasonable doses.

“Methods for the purification of nikkomycins are described in U.S. Pat. Nos. 4,552,954 and 4,315,922. Also, U.S. Pat. Nos. 4,552,954 and 4,315,922 describe various types of nikkomycins, e.g., nikkomycin X, nikkomycin Z. nikkomycin I, nikkomycin J, nikkomycin M, nikkomycin N, nikkomycin D, nikkomycin E. Various nikkomycins are useful in the practice of the present invention. In addition, derivatives of nikkomycins are useful in the methods of the present invention. Additional methods of purifying nikkomycins suitable for use in the present invention are described in Krainer et al., 1987, Anal. Biochem. 160:233-239.” (end quote, relevant to the present invention).

A more recent nikZ preparation is described in Stenland et al., Org. Process Res. Dev. 2013, 17(2), 265-272. This describes a strain of Streptomyces modified to suppress production of nikkomycin X and stressing the strain to increase overall production. Isolation of nikZ follows the principles used in earlier reports, such as those cited just above.

### Example 1—DiW NikZ in Mice

Sustained release oral formulations of many drugs are well known. These are difficult to prepare and evaluate in mass or simple screening, but easy enough to develop commercially if important. This study was designed to get some idea of the impact of spreading out dosing, even at an imperfectly smoothed rate. The results of this study suggest this formulation provides significant clinical benefit, and promise perhaps even more benefit from a commercial extended-release (XR) oral dosage form.

### Methods

### Animals.

Female, 6-week-old, CD-1 mice from Charles River Laboratories were acclimatized for 1 week prior to use in these studies. Mice were randomized to experimental groups, housed in groups of 5 in microisolator cages. The mice were provided sterilized food and acidified water ad libitum, under Animal Biosafety Level 2 standards. All animal experiments were done under an approved protocol of the Institutional Animal Care and Use Committee of the California Institute for Medical Research. All guidelines for animal care and use from the Office of Laboratory Animal Welfare, National Institutes for Health, Washington, D.C., USA, were followed (National Research Council, 2011).

### Organism.

Coccidioides posadasii strain Silveira (ATCC 28868) was used in these studies. The organism was grown and arthroconidia for inoculation prepared as described previously (Clemons et al., 1990). All growth and handling of the organism were done under Biosafety Level 3 containment (Chosewood & Wilson, 2009).

### Dose Formulation, Drug Product, DP.

PO (per oral) “drug in water” (DiW) doses each were prepared in a concentration predicted to deliver the desired dose, on average, to a group of mice consuming an expected amount of water daily. Assuming each mouse consumed water at rates similar to companions in that cage, the water consumed divided across that group of mice allows simple calculation of the amount of ingested drug per mouse. Doses were calculated as nikZ in water at 1.03, 3.06, and 10.3 g/L to give 200, 600, and 2,000 mg/kg/day per mouse. Drinking was as expected, confirming these doses were ingested. A fresh preparation of drug in water was presented daily. Drinking was measured by bottle weight. Medicated water was provided in excess of daily needs, ad libitum. In later studies, a fresh preparation of drug in water was changed twice a week, topping off the reservoir if needed to assure at least somewhat more than a full day supply.

Three therapy cohorts were injected with nikZ IP as an alternative both for comparison and to ensure that at least some animals would receive the intended dose. IP injections were given BID to equal the same daily doses, recognizing that bypassing oral uptake should give higher blood drug levels. Doses were prepared at about 8, 24, and 80 g/L. An injection of 0.2 ml was calculated to deliver the intended the low and middle IP doses, and 0.3 ml for the high IP dose.

The highest nikZ concentration for IP application (1000 mg/kg, BID) was not fully soluble in the chosen water volume, so was injected as a suspension. Immediately after application mice were apathetic and showed ruffled fur. Mice were back to normal within an hour. In the future, injecting a larger volume of the 24 g/L formulation could reach the 1000 mg/kg dose.

As an active control, fluconazole (FCZ) was administered on the same therapeutic schedule (5 days), at 100 mg/kg, q.d. by oral gavage.

A non-treated control in the IP group consisted of injecting sterile water. For a non-treated control in the DiW group, the mice were given normal mouse drinking water.

### Infection Model.

The model of infection used in these studies was that of establishing systemic disease similar to previous investigations (Clemons, K. V., Leathers, C. R. & Lee, K. W. (1983). American Society for Microbiology, New Orleans: F78, p. 395; Clemons, K. V., Leathers, C. R. & Lee, K. W. (1985a). In Coccidioidomycosis: Proceedings of the Fourth International Conference, pp. 149-159. Edited by H. E. Einstein & A. Catanzaro. Washington, D.C.: The National Foundation for Infectious Diseases; Clemons, K. V., Leathers, C. R. & Lee, K. W. (1985b). Systemic Coccidioides immitis infection in nude and beige mice. Infect. Immun. 47, 814-821; Clemons, K. V., Hanson, L. H., Perlman, A. M. & Stevens, D. A. (1990) Antimicrob. Agents Chemother. 34, 928-930; Clemons, K. V., Homola, M. E. & Stevens, D. A. (1995). Antimicrob. Agents Chemother. 39, 1169-1172; Clemons, K. V., Grunig, G., Sobel, R. A., Mirels, L. F., Rennick, D. M. & Stevens, D. A. (2000). Clin. Exp. Immunol. 122, 186-191; Capilla, J., Clemons, K. V., Liu, M., Levine, H. B. & Stevens, D. A. (2009), Vaccine 27, 3662-3668).

The number of c.f.u. (colony forming units, CFU) remaining in the lungs, liver and spleen is determined by quantitative plating of organ homogenates as described previously; these are the primary target organs of infection in this model. (Clemons et al., 1983, 1985a, b, 1990, 1995; Clemons, K. V. & Stevens, D. A., J. Antimicrob. Chemother., 1992. 30, 353-363; Clemons, K. V. & Stevens, D. A. (1994 J. Med. Vet. Mycol. 32, 323-326).

Groups of 10 mice were infected intravenously with about 193 arthroconidia of C. posadasii in a 0.25 ml volume. Treatment was started on day 4 after infection and continued for 5 days.

The inoculum count was determined initially by counting arthroconidia from cultures and ascertained by determining CFUs.

The group sizes were determined using StatMate version 2 (GraphPad Software) to have approximately 80% power to detect differences in survival at the 0.05 level. These group sizes have been robust for determining differences in outcome using non-parametric statistics.

Mice were examined daily. In general, mice did not show signs of distress (such as ruffled fur, apathy, severe agitation) in response to nikZ or FCZ (fluconazole) treatment. The highest IP dose caused some temporary discomfort (see dose section above).

All groups appeared otherwise normal for the duration of the experiment with the exception of the infected, untreated group which showed mild distress after 11 days (5 days of therapy in the therapy groups). All mice survived infection and treatment, and for the duration of the therapy period.

On day 11 post challenge, mice were euthanatized using CO₂ asphyxiation. The number of c.f.u. remaining in the lungs, liver and spleen was determined by quantitative plating of organ homogenates.

### Statistical Analysis.

Comparative survival was analyzed by log rank test and the residual burdens of C. posadasii in the organs were compared using a Mann-Whitney U test using GraphPad Prism (version 3.1).

### Results

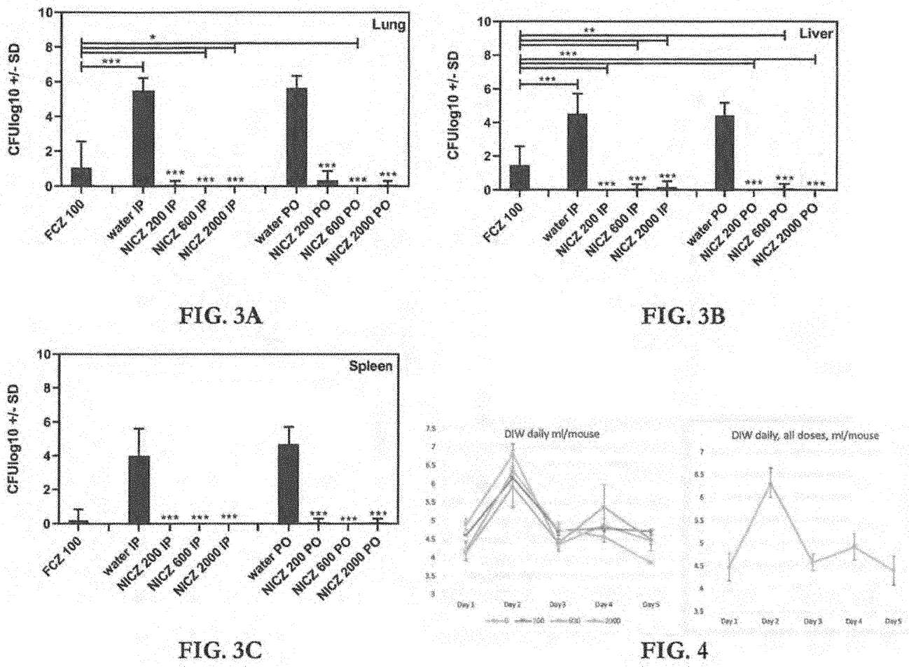

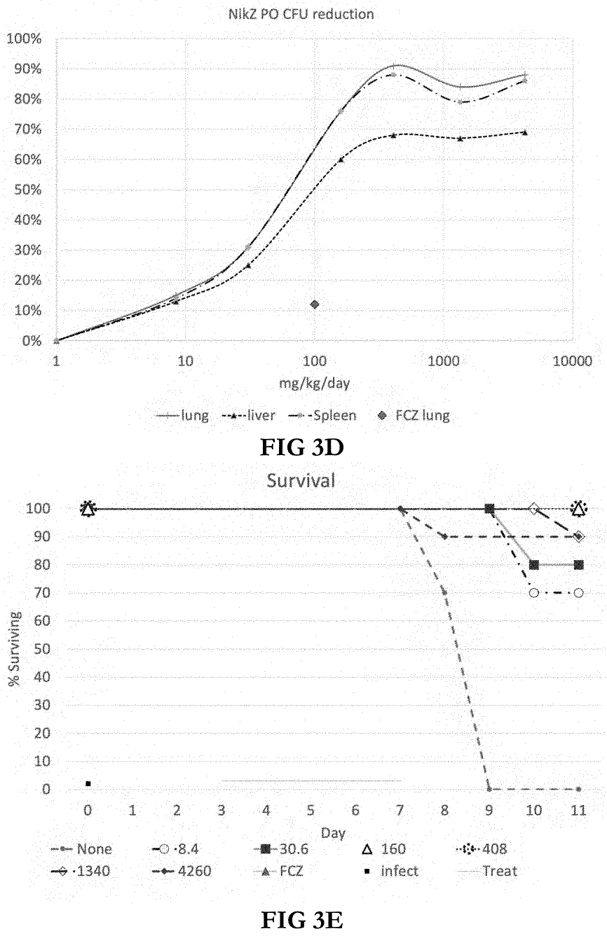

The infection and organ CFU results are shown in FIGS. 3A (lungs), 3B (liver) and 3C (spleen).

FIG. 3A. Fungal loads in livers: t-Test: *p≤0.05, ***p≤0.001. Comparisons without bracket for each group: water vs all other bars. Other comparisons as indicated by the ends of the bracket.
All NICZ doses significantly decreased fungal loads in lungs, independently of the route of NICZ application.
Several NICZ doses were significantly more anti-fungal than FCZ (fluconazole) 100 mg/kg.

FIG. 3B. Fungal loads in livers: t-Test: **p≤0.01, ***p≤0.001. Comparisons without bracket for each group: water vs all other bars. Other comparisons as indicated by the ends of the bracket.
All NICZ doses significantly decreased fungal loads in livers, independently of the route of NICZ application.
Several NICZ doses were significantly more anti-fungal than FCZ 100 mg/kg.

FIG. 3C. Fungal loads in spleens: t-Test: ***p≤0.001. Comparisons without bracket for each group: water vs all other bars.
All NICZ doses significantly decreased fungal loads in spleens, independently of the route of NICZ application.

### Table 1:

Water consumption—listing average drinking (by cage per day within two cages per cohort, scaled to per mouse), standard deviation, and coefficient of deviation for each pair of cages per cohort per day. The changes, particularly on day 2, may reflect the time of measurement varying day to day, with some longer durations between measurements showing more consumption because of more time rather than faster drinking. This will be further evaluated in new experiments. Two outliers are highlighted, likely reflecting an artifact, likely a leak rather than drug or water consumed.

FIG. 4 charts the drinking as reported. The tools are still developing. The data seems sufficient to demonstrate overall drinking patterns, but with detailed information limited only to daily consumption, with no insight into drinking over shorter time frames. Very simple protocol changes are being considered that may provide more detail in future experiments.

Referring to FIG. 4, the rate of drinking among cohorts moved together, with somewhat more inter day variation than might be expected. Recording the time of measurement more carefully should smooth out that data. The respective groups drank similar amounts of water (generally), all approximating each most drinking about 5 ml per day, about as expected. There seems to be no dose-dependent change in behavior, with the note that drinking at the highest dose is a bit lower on the final day, but this could be within measurement variation.

### Mouse Weights (Table 2 Below)

Mice were weighed individually within groups before infection and after therapy, but not serialized by specific mouse, giving only cage level details. Variations in weight before infection were similar to variations at end of therapy. Weight gain in the PO cohorts suggests some dose response in weight gain (nonlinear, inverse relationship to dose, trending to higher weights at larger doses). Limited data suggest at this point watching weight details in the future at other durations, doses, and infection scenarios.

It is possible that mice with the highest doses were healthiest and were eating more. If a 15% weight gain over 5 days is close to normal, perhaps the high dose PO group is showing normal weight gain and all others (infected, with less drug) ate less than normal. This would suggest the high dose group may have been the most comfortable of all groups. The weight gain in the 600 mg/kg/day IP group could reflect the same. Mice receiving the highest dose IP were a bit perturbed. The data is probably too limited and too noisy to draw broad conclusions at this point.

TABLE 2
Mouse weights before injection and after therapy

| | PO 0 | 200 | 600 | 2000 | IP 0 | 200 | 600 | 2000 | FCZ |
|---|---|---|---|---|---|---|---|---|---|
| Start | 25.0 | 24.6 | 23.1 | 23.4 | 23.6 | 23.6 | 23.7 | 23.3 | 24.4 |
| Avg end | 25.2 | 26.1 | 25.5 | 27.3 | 25.4 | 23.5 | 25.7 | 24.1 | 24.2 |
| sd | 1.5 | 2.1 | 1.7 | 2.2 | 1.9 | 0.85 | 1.7 | 1.9 | .55 |
| COV % | 5.8 | 8.0 | 6.6 | 8.1 | 7.7 | 3.6 | 6.7 | 7.9 | 2.3 |
| Weight Gain | 0.14 | 1.57 | 2.41 | 3.88 | 1.75 | −0.08 | 2.02 | 0.8 | −0.27 |
| % gain | ~0 | 5.7% | 9.4% | 14.4% | | | | | |
| % gain/dose | | .03% | .02% | .01% | | | | | |
| mg/kg/day | | 197 | 606 | 1890 | | | | | |

Observing that particularly for the high PO dose, the drinking tapered off a bit on the last day (about 10% less than the day before or for other groups), it is possible that the dosing encouraged the mice to eat more, perhaps reflecting some slight distress or perhaps some degree of avoiding the drug in water. This may be simply an experimental artifact.

### Discussion

This study tested two important novel modalities. First, there seem to be no reports of using nikZ as therapy against injected, disseminated disease. Second, this presents a novel method to administer drug in a semi-continuous manner that is fairly easy to administer.

This showed that rather significant dosing levels can be provided to the test subjects. The doses showed excellent clinical outcomes, essentially eradicating the disease even with only 5 days therapy. Repeat testing will ascertain whether such dosing can be sustained for longer periods, long enough for a clear and useful clinical benefit, and perhaps at lower doses.

There is a concern that an animal too seriously diseased might reduce its rate of water drinking and thus not get the desired drug level through the proposed DiW modality. A group of animals was included with equivalent dosing levels administered i.p. to have at least some animals receiving the dose range of interest. This was expected to give a somewhat higher blood drug level than the oral route, and spread somewhat over time, slower than an IV bolus. All IP doses were effective, despite some minor technical issues with administering the highest dose.

As tested, animals consumed water at a generally normal rate for the duration of the experiment, at all drug levels including the untreated controls. The drinking data will be more informative as new information from new study groups is added.

With a nikZ plasma half-life of about 2.5 hours (roughly similar in mice and humans), traditional pharmacokinetic protocols call for dosing at least every 12 hours, and preferably every 8 hours or even more frequently. To maintain a satisfactory blood drug level throughout the dosing interval, rather large doses are required for TID, and more so at BID. A complication is that uptake after an oral dose shows less than linear blood drug level increases when giving higher doses. Dosing every 12 and even every 8 hours to establish the desired blood drug levels pushes the unit dosing into this inefficient non-linear region. (Nix 2009). This complicates the typical dosing scheme if higher blood drug levels are desired. Frequent dosing is a challenge for patient compliance even for acute therapies, and much harder in chronic therapy.

Hector 1998 demonstrated 100% survival in rats after injecting a fatal dose of Candida albicans when treating with nikZ administered for 96 hours continuously by IV or semi-continuously by s.c. sustained release formulation.

For dose ranges in this Study 1, a first dose selection was low, slightly higher than a typical dose used in nikZ testing against coccidioidomycosis. Early tests of nikZ against nasal infection with Coccidioides spp. typically use nikZ doses of 5, 20 and 50 mg/kg, BID, by oral gavage. (Hector 1990) Taken BID, this older high dose is 100 mg/kg/day. This corresponds to approximately 250 mg BID for a human, which is anticipated to be a moderately effective dose level against Coccidioides spp.

Choosing a low dose of twice this level, 200 mg/kg/day (about 1 g/L), equivalent to 500 mg BID for a human, is anticipated to be a likely range for useful dosing nikZ against Coccidioides spp.

For a high dose, conveniently 10 times higher, a dose already proven well tolerated in toxicology studies, 2000 mg/kg/day was used (about 10 g/L DiW). In the earlier test, mice were given this dose q.d by oral gavage. After 7 days, a thorough workup showed no signs of abnormality or distress in the animals. There are many reports in the literature of dosing at 1000 mg/kg/day in mice or rats.

The test intermediate dose of 600 mg/kg/day (about 3 g/L) was chosen as a typical intermediate step, roughly ½ log order between the low and high doses.

Since the rate of drinking was not predictable in advance, anticipating particularly that sick mice reduce their rate of drinking, the viability of this DiW dosing mode was uncertain.

An important question was not knowing whether mice would accept the drug presented in this form. Taste, pH, viscosity, saltiness, thirstiness, disease impairment—many factors might influence drinking rates. The taste is quite moderate to humans, even at the highest concentrations tested. The mice drank all doses with generally good avidity, from normal mouse drinking water to the highest oral DiW dose presented.

The drinking control and two DiW (drug in water) groups showed steady and generally consistent drinking throughout the therapy, for the 0, 200 and 600 mpk/day groups. In the DiW 2000 mpk/day group, drinking tailed off about 10% on the last day, but the data is coarse enough that this could be simply an artifact. A review of the data shows that the planned dose levels were at least approximately achieved, with a 5% low level (1900) in the 2000 mpk/day group, noting the mice put on weight.

It is interesting to note that all PO groups gained weight, in a nonlinear, inverse dose dependent trend, gaining about 6%, 10% and 16% respectively in the 200, 600, and 2000 mpk/day groups, which corresponds to respectively 0.03%, 0.02% and 0.01% gain per mpk dose. Considering that disease may suppress appetite, perhaps this pattern means that the high dose PO group was close to normal, and weight gain in the others was suppressed.

A variable that is difficult to assess is ancillary loss of DiW water bottle drug source (animals bumping the water apparatus, preparation, and handling challenges). In two measurements there was noticeable extra loss (slip, stopper seating mismatch, corrected quickly but only after some loss). Since the drinking rate (DiW water weight day to day) was for the most part consistent with historical expectations, it seems likely that ancillary losses were not large.

The drug activity (potency) of DiW with aging (possible degradation) of the prepared dose as presented also was uncertain, so the initial plan was to prepare fresh doses twice a day. Daily changes proved to be practical and effective. After further study, DiW changes can be every four days, perhaps longer. This was in a temperate environment of about 20-25° C. Aging may be faster in warmer environments, such as ambient atmospheric conditions in a desert region in the summer.

Historical studies report that nikZ degrades at about 1% per day at pH 4, near the native pH of 3.35 for the HCl salt in manufactured form, when reconstituted in water at a wide range of concentrations. The primary degradation products are safe, but have insignificant pharmacological activity, so degradation is primarily a small loss of activity. After study a single preparation is likely to cover up to 3 days without operator intervention, and likely could be used even longer. This makes dosing over weekends more practical.

Using available tools, monitoring the UV spectra of DiW prepared samples held for many days at room temperature showed minimal change in nikZ, as expected generally, but this for longer than tested recently in detail. UV is not a technique well suited for providing a complete or detailed picture of degradation, so this is only indicative. Testing in an in vitro system showed potency essentially unchanged during 96 hours, at 20-25° C. conditions found in the vivarium. Subsequent testing by hplc confirmed that samples as prepared for mouse dosing degraded about 1% per day to cleavage products NikCz and NikD (breaking at the nikZ amide bond), both non toxic and having no significant if even known biological activity. Refrigerated, this degradation is about 1% per month.

### Study 2

A second study basically a copy of Study 1 but with a much higher disease challenge of 1100 CFU IV showed that doses in mice as low as 8 mg/kg/day showed some improvement (equivalent to fluconazole at 100 mg/kg/day) and 200 mg/kg/day limited disease by 75% (˜5 log orders) after a brief 5 day therapy. This suggests about the same blood drug levels of nikZ are effective against a range of disease severity. Further studies are planned to explore more therapy parameters. Referring to FIG. 3D, the triangles list CFU measured in liver under various dose levels. CFU levels in lung are show by “+” markers, and in spleen by closed circles. The reduction as plotted is as a % of log 10 units of treated tissue vs. levels in untreated controls. Table 3 reports the log 10 CFU counts by dose and organ, with average (10 subjects, mostly), standard deviation, and standard error of the mean for each dose/organ record. The dosing targets were not quite reached, as the animals began to show signs of significant infection before therapy was initiated and were slow to begin drinking (and getting therapy). Untreated subjects showed 7, 7 and 6 log 10 units in lung, liver and spleen respectively. Dosing at 408 mg/kg/day (target was 600) gave pretty much maximal inhibition of fungal proliferation. The responses at the highest two doses were favorable, but not in a monotonic series with the doses up to 408. This is not yet explained. It suggests generally that a dose close to 408 would be sufficient in treating this very severe infection challenge.

Fluconazole at 100 mg/kg/day, qd, gave only slight protection. Extrapolating the nikZ curves, the nikZ equivalents are noted in table 3. In general, all levels of nikZ were more effective than this reference dose of FCZ, and most levels of nikZ were significantly more effective.

Referring to FIG. 3E, all of the FCZ controls (shown in the 100% survival cluster) survived to the end of the brief study period, despite the significant fungal burden noted in Table 3. The nikZ-treated groups showed decreasing losses at 8.4 mg/kg/day (open circle), 30.6 mg/kg/day (closed squares) and 134 mg/kg/day (closed diamond) but all higher doses survived. The untreated controls all failed by day 9.

### Dosing Models

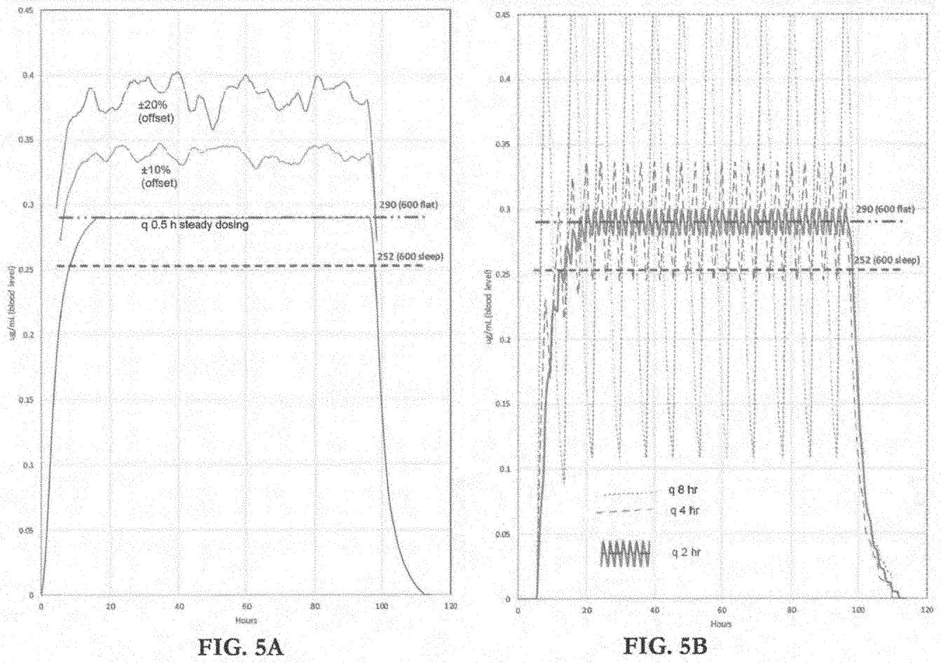

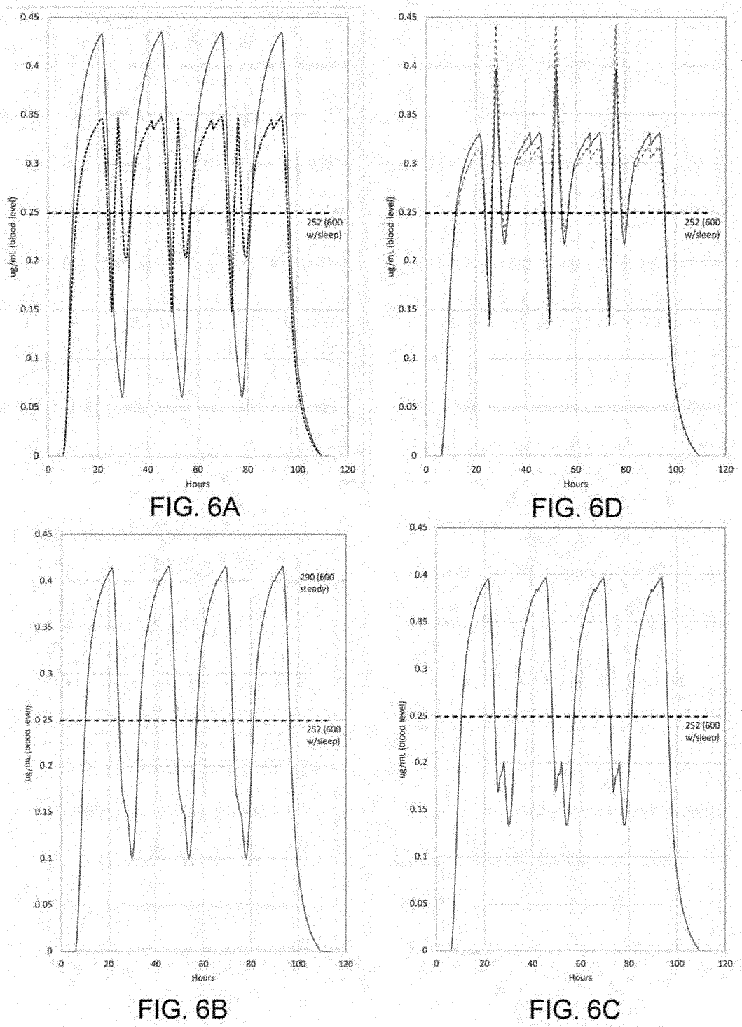

Referring to FIGS. 5A-B and FIGS. 6 A-D, in a model of dividing a dose over 24 hours, delivered at 30-minute intervals, based on reported human PK blood drug levels after a single oral dose of 250 mg, scaled to the dose necessary for each time slice of therapy. Modeling 48 30 minute time units and even dosing gives the pattern of FIG. 5A. Taking doses evenly at 2, 4 and 8 (not 6) hour intervals is illustrated in FIG. 5B. FIGS. 5A and 5B show blood level in μg/mL nikZ levels over 120 hours. In FIG. 5A, a single oral dose is divided into 48 portions, each given at 30 minute intervals repeatedly until stopping at 96 hours (4 days). FIG. 2B on the left panel shows a single human dose of 250 mg gives a Cₘₐₓ of about 2.1 μg/mL at about 2 hours, decaying about 90% at hour 12 (10 hours later), a half-life of about 2.5 hours. In FIG. 5A, the steady state level of 0.29 μg/mL is modeled at 600 mg/day in a 65 kg human (5 mg/kg/day).

This is only a model, as the amount of drinking is more episodic, but the model helps understand one possible pattern of dosing. A human can be trained to stay within this 30-minute time period rate of truing up the rate of ingestion. FIGS. 6A-D include a pause in dosing during sleep, and various scenarios for restoring dosing while awake.

As an alternative model, ultimately disfavored, assuming the mammal will not drink while sleeping, then assuming a waking cycle of 16 hours, the daily dose can be modeled as 32 microdoses over those 16 hours. A model that seems more helpful to flatten drug levels is to make adjustments around sleep patterns, as detailed in FIGS. 5A, 5B and 6A-6D.

Staying on schedule about every 2 hours is enough to give good blood drug level control, but a finer dosing gives even better control.

Consuming drug as microdoses raises blood drug levels in the scale of the half-life of elimination. Thus in 2.5 hours (one clearance half-life) the blood drug level will rise to 50% of the steady state at that dosing rate, and in 7.5 hours the blood drug level will rise to 88% of the steady state.

Each microdose will have its own PK profile of initial absorption, increase in blood drug levels to a Cₘₐₓ at time Tₘₐₓ, and course of elimination, aggregated with historical levels from doses recently taken. Taking these microdoses at a frequency such that the preceding dose has not almost completely cleared will lead to a net rise in drug levels, taken against limits of elimination and all processes well known from study of continuous IV infusions (rate of input, rate of elimination/metabolism, time to steady state, more). The point of frequent microdoses is to add small amounts of drug frequently to give a cumulative blood drug level, preferably in a fairly short period of time.

At steady state with an episodic intake, each microdose will raise somewhat the blood drug level, followed by a decrease from elimination/metabolism, then another rise from the next microdose. These fluctuations depend on the frequency, consistency of dose, and timing of dose (time shifts from the nominal frequency). Averaging and overlapping many microdoses evens out significant small variations in frequency, dose amount and timing nuances.

FIG. 5A illustrates highly steady and consistent dosing, at 30-minute intervals, about 20% of the clearance half-life. For a drug level over time model, human PK data for blood drug levels after a 250 mg dose were used. See FIG. 2B, left panel, open circles. Tₘₐₓ 2 hours, Cₘₐₓ 2.2 ug/ml, half-life about 2.5 hours. This pattern was scaled to the divided dose level (small amounts), stepped over time for each subsequent contributing dose (new input every 30 minutes), then all dose contributions summed at each time point. In this aggregate, blood drug levels rise in 2.5 hours to 50% of the target steady state, just as in IV infusion models. After 7.5 hours (3 half-lives), 88% of the threshold is reached. Infusion models generally assume that 6 half-lives gives 99% of the steady state, so even at 5 half-lives (12.5 hours) the blood drug level is close to steady state. This model predicts that daily dose of 600 mg will give a blood drug level of 290 ng/ml, well above the reported and anticipated 62.5 ng/ml expected MEC (minimum effective concentration). Note that these models are all ratio based, looking at both mouse mg/kg/day dosing and human mg/day dosing, which are not identical but the patterns of accumulation and clearance are the same, proportionally, so modeling either mouse or human inputs will give useful information about the impacts of perturbing a regular pattern. FIG. 5A also illustrates the effect of randomizing the dose amount at each time unit. Adding a randomization of 10%, Cₘₐₓ is 0.30 and Cₘᵢₙ is 0.28, or C_{avg}±0.01 (3.4%). This is illustrated offset for readability, actually centered on 0.29 μg/mL, shown offset centered on 0.335. Similarly adding a randomization of 20%, Cₘₐₓ is 0.315 and Cₘᵢₙ is 0.265 or C_{avg}±0.025 (8.6%). This is illustrated offset for readability, actually centered on 0.29 μg/mL, shown offset centered on 0.385.

FIG. 5B illustrates the effects of dosing frequency. Comparing the essentially flat line when dosing at 30 minute intervals (FIG. 5A, without randomization), FIG. 5B shows a model of even dosing at 2 hour intervals (solid line), 4 hour intervals (dashed line), and 8 hour intervals (dotted line). As shown in FIG. 5B, dosing every two hours, Cₘₐₓ is 0.30 and Cₘᵢₙ is 0.28, or C_{avg}±0.01 (3.45%). Dosing every 4 hours, these values are 0.335, 0.245, ±0.045 (15.5%). Dosing every 8 hours, these values are 0.40, 0.11, or C_{avg}±0.18 (62%).

In a model of relatively consistent dose consumption in 30-minute increments during all time periods, adding a randomization factor of even 15% (for each modeled dose) makes only modest difference in the near steady state average blood concentration. Table 4A.

TABLE 4A
96 hrs.

| Sleep + 4 | % | AUC | Max | Min | mpk unit |
|---|---|---|---|---|---|
| 0 | 0 | 2.37 | 0.099 | 0.099 | 2.04 |
| 0 | 15 | 2.38 | 0.107 | 0.092 | 2.09 |
| 0 | 15 | 2.63 | 0.154 | 0.019 | 3.1 |
| 8x | 2 | 2.33 | 0.119 | 0.051 | 2.46 |

A dosing sequence with less variance from the steady state average can be managed more precisely to give just enough drug to establish blood drug levels needed

During an 8-hour sleep cycle, drug levels fall by almost 90% (about 3 half-lives of 3×2.5 hours). Adding a “Sleep+4” dose at about 4 hours after starting the sleep cycle helps restore blood drug levels. Taking a “2×” double the usual dose within a 30-minute period during waking takes the total daily units from 32 to 34, thus each unit is slightly smaller. Taking instead an “8×” eight times the waking period dose takes the daily units to 40 segments, now approaching the 48 of constant dosing day and night. Table 4B lists a few pharmacokinetic values from this model.

TABLE 4B
48 hrs. detailing boost dose

| Sleep + 4 | AUC | Max | Min | mg/kg/unit |
|---|---|---|---|---|
| 0x | 2.33 | 0.145 | 0.020 | 3.0 |
| 2x | 2.28 | 0.136 | 0.032 | 2.82 |
| 4x | 2.23 | 0.129 | 0.041 | 2.66 |
| 8x | 2.16 | 0.123 | 0.054 | 2.40 |

FIG. 6A show a model including a sleep interval of 8 hours (solid line), plus the effect of a “sleep+4” dose four hours into the sleep cycle (dotted line). FIG. 6A shows this pattern with no boost (solid line) and an “8×” boost (8 units in addition to 32 units during waking hours). FIG. 6B shows a “2×” boost (2 at 2 am+32 during waking). FIG. 6C shows a “4×” boost. FIG. 6D shows “10×” (solid line) and “12×” (dashed line) boosts.

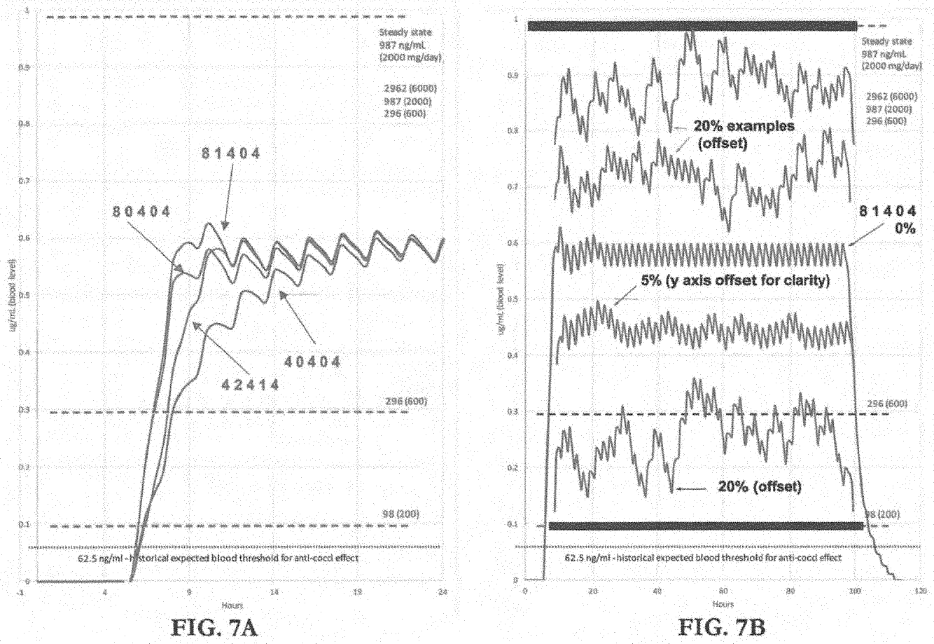

FIGS. 7A and B shows the impact and benefit of a loading dose. This models taking an initial dose at 6 am. FIG. 7A illustrates in detail the effect of various patterns of early dosing over the first 18 hours out of a 96 hour dosing duration. “4 0 4 0 4” means 4 48^{ths} (½^{th} or a 2-hour dose unit) followed by 0, 4, 0, 4 over periods of 4 48^{ths} which is 12 steps a day—every 2 hours. Adding an extra 2 48ths ( 1/24^{th}) at hour 6:30 and an extra 1 at hour 7:30 (4 2 4 1 4) approaches the peak level at 10:30 am, a level reached at only 6 pm (18 hours) without the boost. This boost is needed only in the first 2-hour cycle. In a different pattern doubling the initial dose (8 0 4 0 4), then staying with the standard pattern boosts the initial rise to about 90% of the steady state peak at 8 am, just two hours after initiating dosing, and 100% of the steady state pattern at 4:30 pm. This is likely the easiest pattern for patient guidance and compliance. In a final example, the steady state pattern can be approached even faster by adding one more intermediate dose (8 1 4 0 4). The primary curve is centered on 592 ng/mL, for a dose of 1200 mg per day in a 65 kg human, as in FIG. 7B.

FIG. 7B illustrates the effect of a loading dose over the full 96 hours of dosing and washout. The primary curve is centered on 592 ng/mL, for a dose of 1200 mg per day in a 65 kg human. Dosing every 2 hours gives the tight pattern that will be very useful. This figure also illustrates adding a random 5% variation into each dosing period (offset arbitrarily for clarity, shown centered on around 452 ng/mL) and three examples of including a 20% variation (various offsets, centered around about 230, 730, and 870 ng/mL).

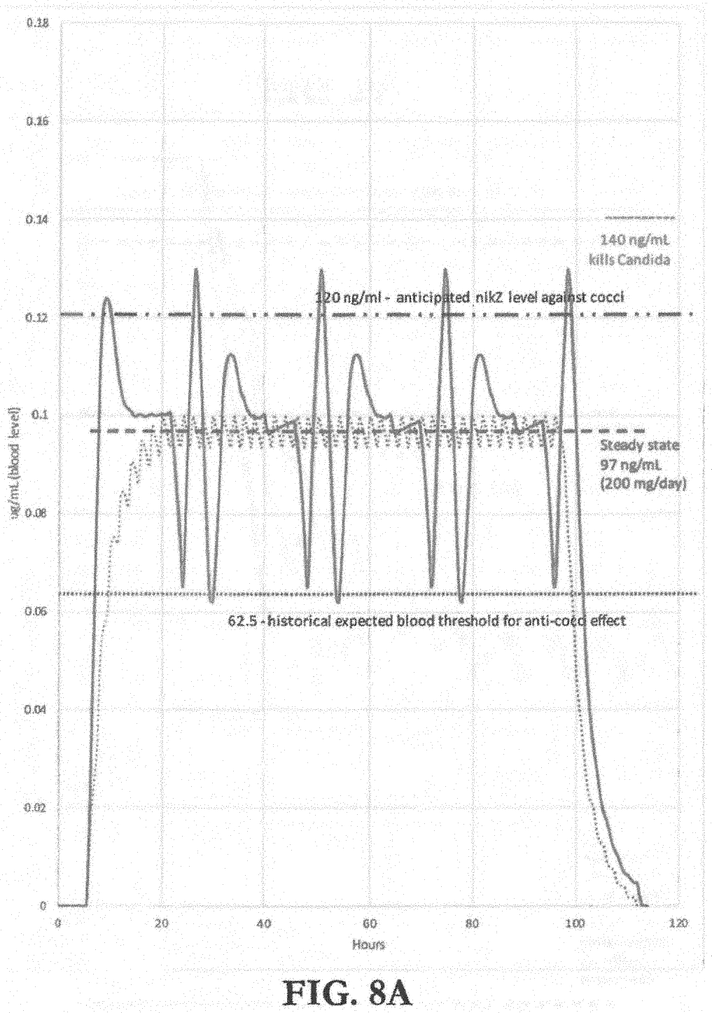

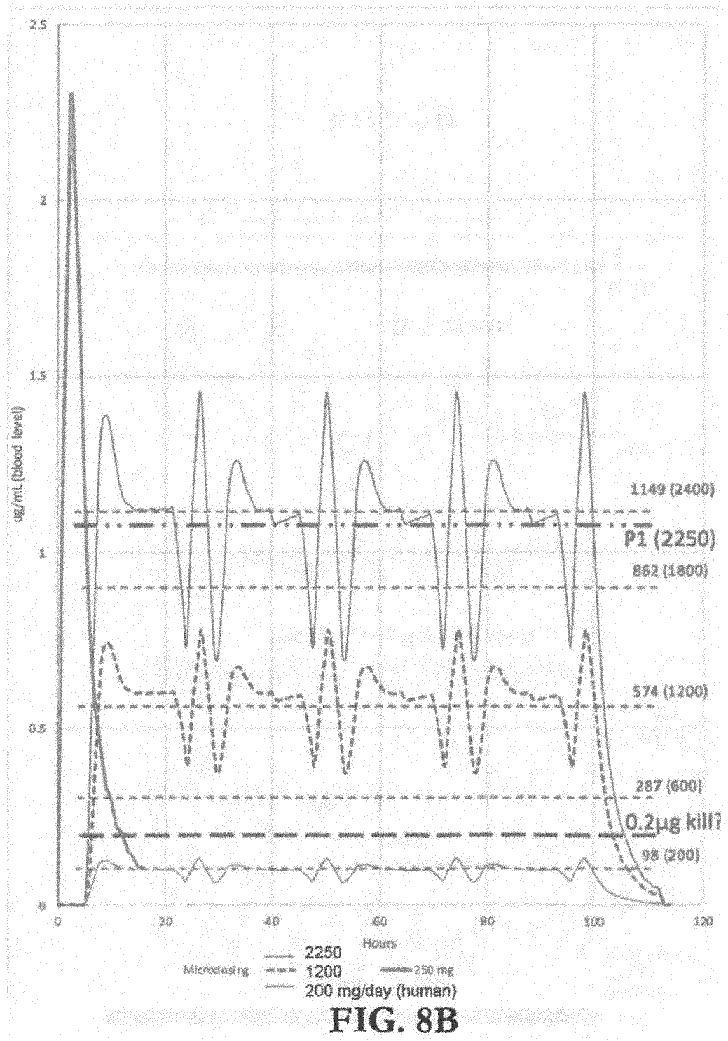

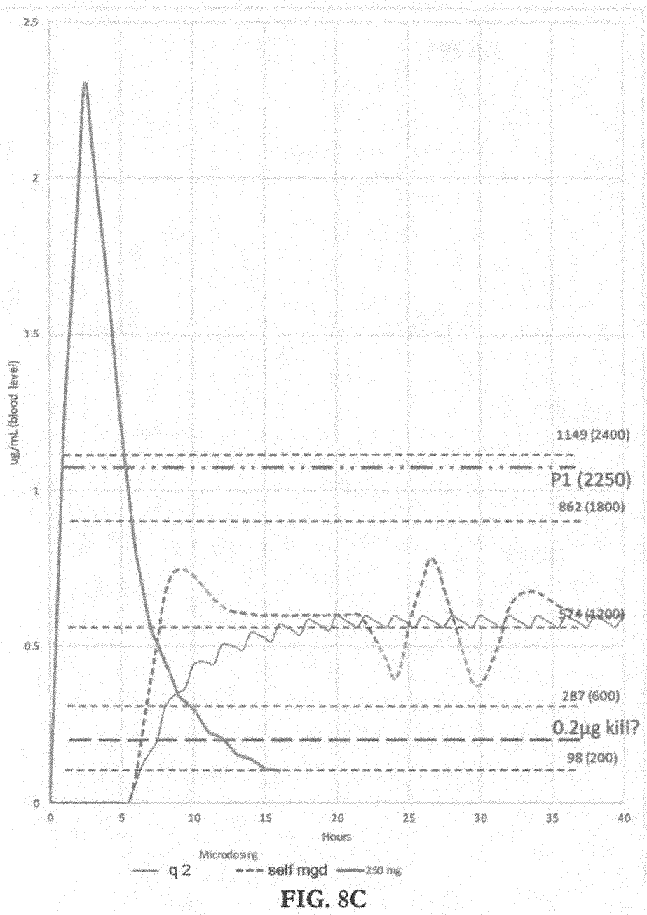

FIG. 8A illustrates another pattern working in many elements from this invention. This example is based on 200 mg per day, in a pattern that gives blood drug levels almost always above 62.5 ng/mL, a level proposed sufficient to kill the VF fungus. This may represent a minimum effective dose. Most estimate are that this is only 40% of a required minimum dose, but q. 12-hour dosing gives very wide blood drug level changes, so this may be testable with the more stable blood drug levels anticipated from microdosing. This may well prove to be enough to treat mild disease. A dose of 200 mg/day in a 65 kg human is 3.08 mg/kg/day. Scaled to a mouse dose (a factor of 8-10) gives 25-31 mg/kg/day in a mouse, well represented in FIGS. 3D and 3E by the 30.6 mg/kg/day dose which indeed suppressed infection noticeably and gave 80% survival against a very high inoculation challenge.

The dosing unit is every 30 minutes. For a loading dose, starting at 6 am on day one, counting dosing units starting from midnight, this is 12 dosing units missed. Guidance is “when behind, consume half the distance to the goal, to a maximum of 5 units (2.5 hours)”, so at 6 am (12 time units) consume 5 drug units. At the next time slice of 6:30 (13 time units), this is still 13−5=8 units behind, and half the distance is 4 units so take 4 units. At 7 am (14 units into the sequence), 9 units have been consumed, 14−9 is 5 units, so half the distance is 2.5 units, so take 3 for a total of 12 units. At 7:30 (15 units), 15−12=3 units, /2=1.5 so take 2. At 8:00 (16 units), 16−14=2 units. Take the 2 units and at 8:30 everything is synchronized. This drives a bit of overshoot in blood nikZ level, to about 120% of the mean. This loading sequence is 5+4+3+2+2=16 dose units which is 8 hours worth. A TID q. 8-hour dose would be the same amount. Compare that single q. 8-hour dose spread here over 2.5 hours by microdosing. A second single TID dose would lead to a significant blood level spike and tapering, contrasted here to continuing to take small doses every 30 minutes by the microdosing method, maintaining a fairly steady blood level at least during waking periods once the early morning stepped dosing drives blood levels to a C_{average} with only small variations.

The guidance listed here is only a model, to map and understand the potential impact on blood drug levels. Since nikZ is quite safe, the actual consumption pattern can be varied significantly. This after all is only an imperfect model to suggest what could be improved with a formal XR dosage form.

During waking hours, the blood drug level is mostly around 105% of the mean.

At sleep time, blood drug levels drop as dosing ceases during sleep. At the “sleep+4” dose (four hours after going to sleep), a 4-hour dose (8 dose units) exceeds the guidance of “5 max” but is well within safety limits. The patient can take 8 units at once, or take 5, wait 30 minutes and take 3 more. Even easier, the patient can take a 3-dose unit (1.5 hour) advance dose before sleeping, then at “sleep+4” time take (8−3=5) dose units.

In the morning, blood drug levels are again reduced. Assuming dosing starts at 6 am, the pattern of the initial day is repeated, with the variation that the 2 am dose covered the gap from 10 pm to 2 am, so the gap at 6 am is 4 hours (8 units). Half of this is 4 units, so take 4 units, and continue the pattern of “half the distance to the goal” until back in sync with the schedule.

FIG. 8B illustrates this dosing pattern scaled to 1200 mg/day (dashed line, human) and 2400 mg/day (solid line). For perspective, a line with a mean blood drug level of 1125 ng/mL represents the dose of 2250 mg already tested in human phase 1 trials. That trial used doses of 750 mg every 8 hours (compare FIG. 5B dotted line q. 8 hour dosing, 600 mg/day). FIG. 8B also illustrates a single tablet of 250 mg (tall, single peak to 2.25 μg/mL). Note that microdosing levels from a fairly high dose of 2400 mg/day has a mean about half the peak from the single 250 mg dose, and stays within about 20% of that blood drug level mean. The 750 mg single dose should peak at about 4.5 μg/ml (see FIG. 2B 500 mg, open circle, lowest in the right panel, and 1000 mg, open squares, middle height in left panel). Compare also FIG. 2A, with a peak at 5 μg/mL in a continuous intravenous infusion, and note also the maintenance level of 170 ng/mL, corresponding to microdosing of about 350 mg/day (human). See FIG. 5B C_{average} 290 ng/mL from 600 mg/day, scales to 352 mg/day for 170 ng/mL (170/290=59%, *600). A blood drug level of 170 ng is ˜340 μmolar nikZ.

FIG. 8C is an expansion of part of FIG. 8B, at 1200 mg/day (human daily dose), focusing on the first 40 hours. This is shown as a dashed line, centered on the (1200) line at about 0.6 μg/mL. FIG. 8C includes a modeled blood drug level curve without loading dose modifications (thin solid line), which also reaches a steady state C_{average} at about the same 0.6 μg/mL.

Note that FIG. 2A is measured from continuous intravenous infusion into a rat, but this still illustrates a tolerated level, maintained for 96 hours. This suppressed a different fungus so that all subject rats survived a fatal intravenous dose of fungus, the virulent B311 strain of Candida albicans.

This suggests that microdosing even at fairly high daily levels is always well below known safety levels.

The main point of this is that the oral microdosing method works well with some variance in the peaks and troughs of blood drug level. The longer the constant dosing can be maintained the better for keeping blood drug levels closer to the average. If a sleep period can be interrupted for an early morning dose, a dose of any size will help. Taken about 4.5 hours after starting sleep is well suited to moderating swings in blood drug levels. A dose equivalent to 4 hours at the waking dose pace will smooth out the levels significantly (“8×” in Table 4B). Taking this even quickly will be well below uptake saturation levels. For example, at 200 mg/kg/day, 4 hours equivalent of 20 (16 waking hours plus this “4”) is 20% or 40 mg/kg for that 8× “Sleep+4” dose. In a human of 70 kg, this would be 240 mg, a dose comfortably in the linear uptake range (unit doses <750 mg in a human). At 4 g/L, this would be 60 ml, about 2 imperial fluid ounces.

In one preferred embodiment, drug is supplied as a powder, such as in a sachet or packet, as a daily dose. A human dose can be conveniently dissolved in about 120 ml of water (4.06 fl. oz), which divides conveniently into 2.5 ml (0.5 teaspoon)/30 minute time unit. At 2 g/day, a significant dose indicated for moderately severe disease, this gives a drug concentration of 16.7 mg/ml, which will dissolve readily. Human doses are anticipated to be about 0.65 g/day to 2 g/day and perhaps higher when treating rather severe disease. Other water amounts and concentrations are easy to understand and choose. The point is to dissolve the daily dose in a useful amount of water, and then drink that water in as regular a pace as convenient. A calibrated container makes it easy for the patient to keep track of how much water/drug has been consumed. In a preferred embodiment, the patient records consumption information.

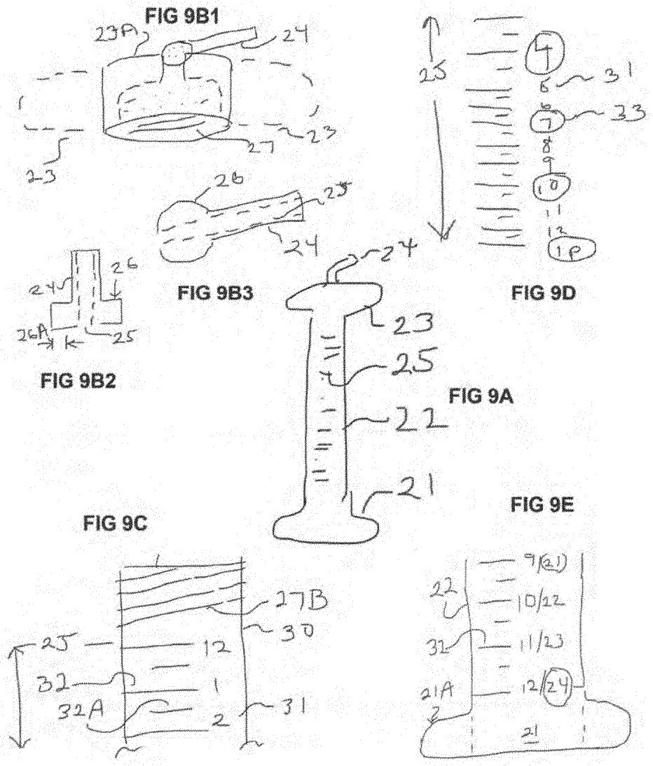

Referring to FIG. 9, a water bottle 22 is calibrated with a scale 25 of 48 marks for half hour periods for the course of a day with major marks 32 for every hour and optional minor marks 32A for half hours. According to one preferred embodiment, hours are labeled for 0 (or 12) (midnight), 1, 2, 3, 4 am . . . 12 p, 1 p, . . . 11 p, 12 p (12 pm=the bottom of the bottle). In one pattern, every third hour 33 is highlighted as a reminder to the patient to both log activity and to keep current with dosing to the time of day. Hours 31 are marked to facilitate reading fluid levels. Any chemist will recognize this a form of graduated cylinder. A widened base 21 provides stability, with flanges 21A. In one preferred embodiment a screw-on cap 23 is designed with some aesthetic balance with the form of base 21. A threaded portion 27 in the cap 23 mates with a threaded portion 27B on water bottle 22. A gasket (not shown) helps with sealing cap 23 to bottle 22. Cap 22 can be fitted with a drinking tube 24. Cap center 23A has channels to support rotating drinking tube 24 around hinge cylinder 26, with extensions 26A fitting into corresponding securing portion (not shown) of cap center 23A. Channel 25 can be cylindrical within drinking tube 24 to provide communication of fluid from within water bottle 22 to the consumer (not shown). Such water bottle fittings 23-26 are very familiar to athletes and widely used athletic water bottles.

This can be marked with a dual scale, one starting at “0” (for first dose of the day) and the other at “6 am” (for example). Including a printed portion that will accept an erasable marker (typical on laboratory beakers) will enable the user to note the actual time of starting dosing on a given day, which could be quite different from 6 am. A write-on strip, or multiple write-on regions can be provided so a user can conveniently set out a schedule convenient for them. For example, a patient may prefer to organize the sequence to start at 1 am, so each hour would be one higher than the standard presented. On a write-on strip, the patient can mark their preferred time frame.

The user is encouraged to keep a simple log of dose rates, organized on a 24-hour day, noting the time of the first dose, the amount consumed by some convenient hour mid-morning, mid-day, mid-afternoon, early evening and late evening, and bedtime. This will provide useful data for the clinician, and importantly will remind the patient to be attentive to consumption and to “catch up” at least every 3-5 hours if needed. From the model above, it is evident there is some flexibility in the mid-sleep period dosing. If doses are prepared for midnight to midnight in the bottle of FIG. 9, a bedtime dose should include any remaining for the day, and after preparing the next day's dose, to some portion to about the expected mid-sleep period. Assuming arguendo that the patient sleeps at 10 and arises at 6, consuming a dose to midnight and then to about 2 am for the next day before bed gives s good drug load for initial sleep. On waking, for example at 6 am, doses for 4 am through 6 am should be consumed. One useful pattern is “half the distance to the goal”. At 6 am, 4 hourly units are due for consumption (2 am to 6 am). Consuming half that is two hourly units (2 am to 4 am). After a 30-minute break, the remaining detriment is 2.5 hours (4 am to 6:30), of which half is 1.25 hours to drink. After another 30 minutes, the normal pattern is close enough so just drink to the 7 am mark and continue from there at the hourly pace.

The model suggests a fair degree of tolerance in pacing. There is definitely time to do tasks for even 4 hours without dosing and catch up when possible, without serious detriment to the overall protocol. “Truing up” every 2 hours should keep blood drug levels within a <3% band, which is preferable to the extent convenient. Truing up every 4 hours should keep levels within a 15% band.

A variety of fitness software “apps” in general circulation are structured to help track a user's daily activity, such as exercise periods, pulse, and other physiological parameters. These are frequently designed to run on a user's smart phone. A number of motivational systems use “gamification” to encourage the user to perform better against their own historical record, against an aggregate profile of typical users, or against other current users going through the same regimens, among other comparators. Some include reward systems. These adapt nicely to this invention as a way to both help incentivize the patient to stay on therapy, to look to keep levels as steady as convenient, and to improve over time.

A range of dosing patterns gives acceptable and fairly consistent blood drug levels. One pattern simply divides the day into 48 segments of 30 minutes. Guidance here is to take drug every 30-120 minutes as much as possible, with finer grained synchronization (30-60 minutes) preferred. After a gap in dosing, particularly for sleep, “catch up” by taking missed doses within limits: A gap of 4 hours (after a boost dose) is 8 30-minute time units, with a 9^{th} unit for the new time point. Consume “half the distance to the goal” or 4.5 units. At the next 30-minute time point, there is a deficit of the new time point+4.5, or 5.5, so take 3 units. After 3 or 4 dose time points the missed doses should be consumed and dosing will be caught up to the current time of day. Pharmacokinetic modeling suggests that taking more than 2.5 hours of dose value at any one time is all that is needed to return blood drug levels to steady state, and that is after a gap of at least 4 hours.

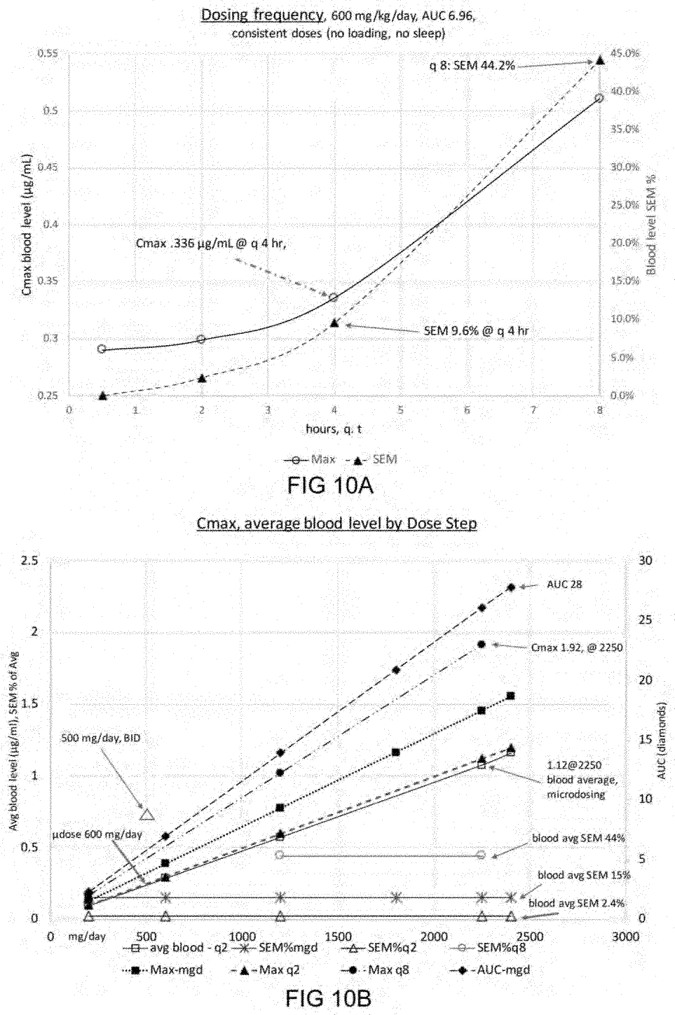

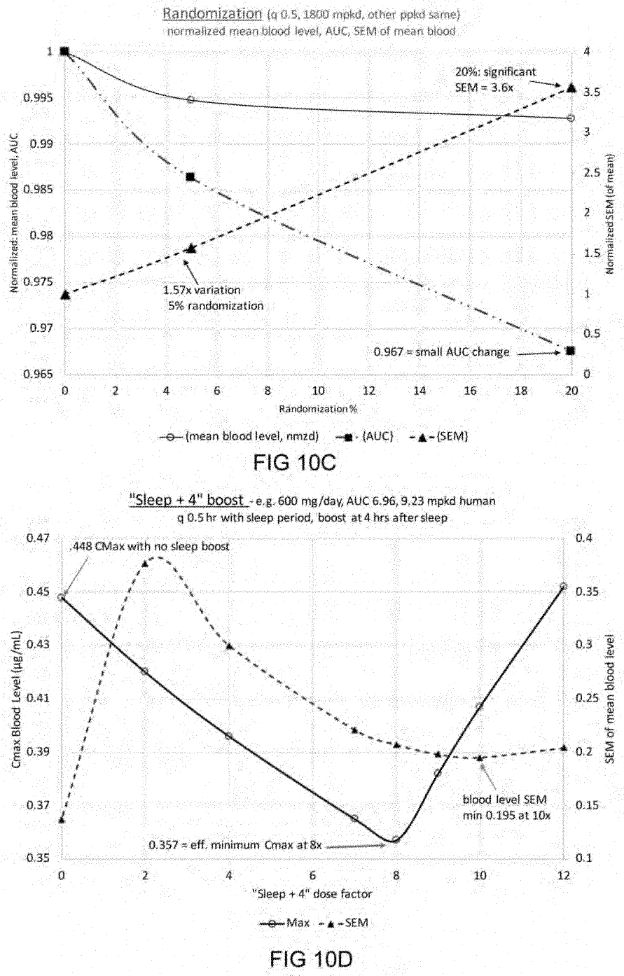

Referring to FIG. 10A, modeling a fixed dose changing dosing frequency shows a significant increase in Cₘₐₓ and SEM (standard error of the mean) in blood drug levels when the time between doses goes above q. 2.5 hours (one clearance half-life), degrading badly (increasing) at q. 4 hours and higher. The abscissa is the frequency of dosing—“q” (every) 0.5, 1, 2, 4 through 8 hours. The left ordinate is Cₘₐₓ (Cₘₐₓᵢₘᵤₘ) of nikZ blood levels in μg/mL, ranging to 0.55 μg/mL (550 ng/mL) in this FIG. 10A. The right ordinate is the standard error of the mean (SEM) for the blood level, in percent of the range of blood level values over time compared to C_{avg} (C_{average}). FIG. 10A illustrates a model of dosing at 600 mg/kg/day (in mice), with an AUC of 6.96, with consistent doses, with no loading and no sleep. This dosing pattern is illustrated in FIGS. 5A and 5B and discussion above. The solid line with open circles charts Cₘₐₓ according to this model, in μg/mL nikZ in blood, Y axis on the left. The dashed line with filled triangles is the SEM, in % variation in blood levels, see right Y axis. At 4 hours, Cₘₐₓ is 0.336 μg/mL, 66% of the Cₘₐₓ of 0.511 μg/mL in a q. 8-hour dosing sequence. A significant impact when changing frequency from q. 4 to q. 8 hours. Note the Cₘₐₓ of 0.29 at q. 0.5 hours is 57% of q. 8 hours, but 86% of Cₘₐₓ at 4 hours and 97% of the Cₘₐₓ at q. 2 hours. For SEM of 9.5% at q. 4 hours, this is 22% of the SEM of 44.2% at q. 8 hours. The change in SEM from q. 0.5 to q. 2 hours is small.

Referring to FIG. 10B, the AUC under microdosing is lower than TID dosing. AUC using TID dosing is lower than AUC using less frequent dosing, not shown. In summary, microdosing gives fairly to very precise blood drug levels and very little extra drug is given or wasted. The abscissa is dose of mg/day (human, from Nix 2009 single ascending human doses, and modeling against those dose levels). The right ordinate (“y”) axis ranges from 0-30 for AUC in μg-h/mL (diamond markers, dashed line, “AUC mgd”), used only in the dashed line with a highlight note at AUC 28 for a dose of 2250 mg/day. Compare a measured 40.7 μg-h/mL for a single dose of 2000 mg (Nix, 2009), extrapolated to 92.8 μg-h/mL if uptake and clearance remained proportional to the low dose values, as discussed in Nix 2009. The current model shows that AUC is decreased considerably with the managed dosing protocol, reducing AUC by 70%, reflecting a savings in drug consumed and patient exposure to the drug.

The left ordinate axis ranges from 0-2.5, relevant to three curves charting the SEM of blood levels (% of average) and to C_{average} (μg/mL) for some curves. A managed (“mgd”) dosing scheme includes sleep periods as described in connection with FIG. 10C. The standard error of the mean (SEM) variation in blood level around C_{average} is 2.4% for dosing q. 2 hours (open triangles). This is almost flat, or 0% for steady state. This variation is independent of dose, thus charting a flat line across doses. The SEM variation in blood level is much higher at 44% for dosing q. 8 hours (open circles) and also independent of dose. Compare FIG. 10A closed triangle, SEM at q. 8 hrs of 44.2%, rounded to 44% in FIG. 10B. For the managed dosing pattern (“mgd”, asterisk symbol), the SEM is higher than for Q2 dosing but lower than for q. 8 hour dosing, and again dose independent.

C_{average} for dosing at q. 2 hours reaches 1.12 μg/mL at a daily dose of 2250 mg/day (human), divided for dosing q. 2 hours (open squares, solid line). Cₘₐₓ for this dosing at q. 2 hours is only slightly higher, consistent with the 2.4% SEM (closed triangles, dashed line). The C_{average} and Cₘₐₓ are dose dependent, linearly increasing with increasing dose. Cₘₐₓ is much higher at 1.92 μg/mL if the same daily dose of 2250 mg/day is divided only into 3 portions (TID, dosing q. 8 hours, closed circles, dot-dash line). Cₘₐₓ for the managed dosing is intermediate between q. 2 and q. 8 dosing (solid squares, dotted line).

FIG. 10C illustrates variable amounts of “sleep+4” single dose consumption, which is illustrated in FIGS. 6A-6D. This FIG. 10C shows that about 8-10× gives the most effective drug utilization and managed blood drug levels. This means a “sleep+4” unit dose of 8 (to 10) times the standard unit hourly dose (32 doses in 16 hours). In this instance, an 8× boost is basically a 4 hour period dose (8 30-minute dose units) taken at once (or over 30 minutes) half way through a sleep cycle of 8 hours. This would make a total of 40 dose units over 24 hours, dividing the daily dose into 40 portions to choose the unit dose. A 2× boost would be 2 extra units, or 34 units over 24 hours. A 10× boost would be 10 extra units, or 42 daily dose units. Many patterns are possible.

In FIG. 10C, a Cₘₐₓ of 0.448 μg/mL (left ordinate) is seen with no sleep boost, decreasing in almost linear fashion as the sleep boost component increases by factors of 2× to 8× (abscissa) then rising again as the boost component is made a still larger component of the daily dose sequence. The variability of blood level changes (SEM, right ordinate scale, to 0.4 or 40%) (dashed line) increases sharply with small “sleep+4” doses, then falls off to relatively consistent variability between 8× and 12×, with a minimum of 19.5% at about 10×.

FIG. 10D studies the effect of randomization. The model is built around changes in dose, but small changes in time give generally the same effect. A randomization of 20% has a noticeable effect. A randomization of 5% has very little effect. Even at 20%, this microdosing is far superior to QID or even q. 4 hour dosing.

This is a model around 30 minute dosing, as discussed above. See FIG. 5A, 6A. This gives an almost flat blood level once steady state is reached. 1800 mpkd is mg/kg/day, a mouse dose level, rather high. As this is a model, uptake saturation is not considered. The model looks at changes to steady state blood levels after steady state has been established, with no changes in dosing with sleep or for other factors. The abscissa is the % of randomization. The left ordinate is AUC in μg-h/mL or normalized mean blood level (C_{average}), in μg/mL. The normalized mean blood level (solid line) decreases with increasing randomization but even at 20% randomization is more than 99% of the level without randomization. The AUC changes some, but still is less than a 4% change at 20% randomization. The right ordinate is the normalized SEM. Compared to FIG. 10C, SEM changes in FIG. 10D are much larger, increasing by 57% with a 5% randomization where in FIG. 10C a 38% change was seen at the 2× sleep boost dose, and about 20% with larger sleep boosts. Returning to FIG. 10D, an SEM of 3.6× (260% increase) is seen at 20% randomization. While these are all only models, these could suggest that changes in blood level through sleep periods with even small boost doses could be roughly comparable to a less than 5% randomization of a steady state input.

General guidance to the user/patient is to take “a few sips” as often as they like, at least about every 60-90 minutes and preferably every 30 minutes, to keep consumption on track with the marked units for the hour of day.

The current expectation is this will be repeated for some days. For patients with mild disease, there is a hope but need to test whether the strong effect in mice in less than 5 days will mean human therapy can be as short as 7 days. For more severe disease, therapy may well need to continue for months.

For clinical trial purposes, at least some particularly sick patients will put up with the inconvenience of this dosing form. If clinical results warrant, there will be motivation to prepare a typical commercial XR extended-release oral dosing formulation. These are available from several vendors and take about 6-9 months to develop and release.

This microdosing form is particularly convenient for humans who may have trouble swallowing an oral tablet or capsule. This is also convenient for dosing animals of most any sort that require therapy. For animals with multiple water sources, or multiple animals sharing the water source, it may be preferred to isolate the sick animal so as to provide a dedicated water supply, or to simply treat other animals with some non-toxic drug. For example, for monkeys in a cage, where 2 of 3 are ill, a shared water supply would conveniently get drug to the sick animals with essentially no risk to the healthy animal. If indeed the rapid clinical benefit observed to date extends to wider treatments, the duration of this DiW presentation will be limited.

### Calculation of Dose Rates

In order to attain a desired minimum effective concentration (MEC) of nikkomycin, e.g., nikkomycin Z, (or other chitin synthesis inhibitor) in the plasma of a subject mammal, it is necessary to calculate the proper rate of infusion (for continuous intravenous administration) or rate of release (for sustained release formulations) of nikkomycin, e.g., nikkomycin Z, for the given subject mammal. This is discussed in good detail in Hector 1998, section 5.4, Col. 9, line 8 to Col. 10, line 29, incorporated here by reference.

Hector 1998 noted that generally, the MEC should be maintained for a period of three to four days against Candida spp. and thus the continuous intravenous infusion or other method of continuous delivery should be for a period of at least three to four days. For Coccidioides spp., this same period of at least three to four days may be sufficient for some instances of disease. The time course and blood drug levels appropriate for treating other fungi need to be studied. Given the high safety of nikZ, there is an expectation such levels can be found to effectively treat many pathogens.

Since portable IV infusion systems are readily available, it is medically convenient to continue infusion for days or even for months if medically indicated.

In some cases, monitoring of the course of infection will dictate that treatment should be for a time somewhat less or somewhat greater than a few days. i.e., those infections that respond quickly will require less time; those that are more refractory will require correspondingly greater time. It is generally expected that treatments of at least several weeks will be common. For many drugs used against Valley Fever, an early therapy may be IV, then a “step down” (often a lower dose) which preferably is oral. There are many variations on these patterns. As each individual will likely respond in an individual way to the therapy and the disease will respond also in a way corresponding to the nature of the specific disease in that patient, clinical practice should start to identify useful patterns and duration.

The microdosing method of this invention can be used for long term therapy. The expectation is that if the therapy proves useful at all, this will provide incentive to invest in developing an XR oral dosage form. The interim use of the microdosing will be a productive effort. Microdosing can be useful to other drug developers as they work out the pharmacokinetics of an approximation of zero order release oral formulations and assess the potential value of preparing such a formulation.

It is well within the ability of one of ordinary skill in the art to determine the proper time course and pattern of treatment.

The present invention is not to be limited in scope by the specific embodiments or specific examples described herein. Indeed, various modifications of the invention in addition to those described herein will become apparent to those skilled in the art from the foregoing description and accompanying figures. Such modifications are intended to fall within the scope of the appended claims. The examples given should only be interpreted as illustrations of some of the preferred embodiments of the invention, and the full scope of the invention should be determined by the appended claims and their legal equivalents.

Various publications are cited herein, the disclosures of which are incorporated by reference in their entireties.

What is claimed is:

1. A method of providing a water-soluble drug in drinking water, comprising
dissolving a drug in water (DiW), and
providing the DiW in a form convenient for a mammal to drink, (freely, in excess).

2. The method of claim 1 wherein the drug is an antifungal drug.

3. The method of claim 2 wherein the drug is nikkomycin Z.

4. The method of claim 1 further comprising providing DiW in excess of daily consumption, made freely available, so as to permit drinking ad libitum.

5. The method of claim 1 further comprising dissolving the DiW at a concentration expected to give a selected dose in the amount of water typically consumed in the course of 24 hours.

6. The method of claim 5 further comprising choosing the selected dose from a range expected to give a favorable clinical benefit against a disease.

7. A method of providing a dose of drug so that portions can be taken during the course of a waking period, the method comprising:
preparing a reservoir of drug,
providing access to the reservoir so a human or animal can ingest the drug in portions at corresponding times over the course of a waking period, thereby dividing the dose of drug taken across respective multiple portions of drug over time,
providing a reservoir sized to encourage ingestion of a drug in approximately the quantity desired for daily therapy.

8. The method of claim 7 wherein the reservoir of drug is in drinking water.

9. The method of claim 8 wherein the concentration of drug is selected to provide a selected daily dose in a typical amount of drinking water consumed by the human or animal.

10. The method of claim 8 wherein a daily dose for a human is dissolved in a measured amount of water and the human is encouraged to drink the drug in water at a rate to just consume the full dose over the course of 24 hours.

11. The method of claim 10 wherein the human is encouraged to maintain a pace of drinking that stays close to the projected dose every four hours or more frequently.

12. The method of claim 10 wherein the human consumes any remaining daily drug before bedtime.

13. The method of claim 10 wherein the human on waking consumes portions of the daily dose allocated to the sleep period and not yet consumed, with the general guidance of “half the distance to the goal” every thirty minutes until catching up to the regular schedule of 1 12th of the daily dose every two hours.

14. A method of dosing wherein a drug is made orally available at a repeat time such that the minimum concentration of blood drug level is more than 50% of the average blood drug level during periods of repeat dosing not including sleep periods or breaks.

15. The method of claim 14 of dosing wherein a drug is made orally available at a repeat time less than approximately 0.8 to 2 times clearance half-life time.

16. The method of claim 15 of dosing wherein nikkomycin Z is made orally available and consumed about every four hours or more frequently.

17. The method of claim 16 of dosing wherein nikkomycin Z is made orally available and consumed in a pattern to stay close to a rate of truing up every two to four hours with a projected rate of consumption.

18. The method of claim 17 of dosing wherein nikkomycin Z is made orally available and consumed in a pattern to stay close to a rate of truing up every two to four hours with a projected rate of consumption, with a pre-sleep period adjustment of consuming drug portions allocated for 2 to 4 hours of anticipated sleep, then within 90 minutes after waking consuming drug portions allocated for hours of sleep after the pre-sleep consumption.

19. The method of claim 14 of dosing wherein a drug is made orally available at a repeat time such that the minimum concentration of blood drug level is more than 85% of the average blood drug level during periods of repeat dosing not including sleep periods or breaks.
